(19) United States
(12) Patent Application Publication
(10) Pub. No.: US 2018/0147265 A1
(43) Pub. Date: May 31, 2018

(54) METHODS RELATING TO THE TREATMENT OF COLITIS AND INFLAMMATORY BOWEL DISEASE

(71) Applicant: CHILDREN'S MEDICAL CENTER CORPORATION, Boston, MA (US)

(72) Inventors: Denisa D. WAGNER, Dover, MA (US); Naamah L. ZITOMERSKY, Brookline, MA (US)

(73) Assignee: CHILDREN'S MEDICAL CENTER CORPORATION, Boston, MA (US)

(21) Appl. No.: 15/572,270

(22) PCT Filed: Jun. 17, 2016
(86) PCT No.: PCT/US16/37988

§ 371 Date: Nov. 7, 2017

Related U.S. Application Data
(60) Provisional application No. 62/182,087, filed on Jun. 19, 2015.

(51) Int. Cl. A61K 38/48; A61P 1/00; A61P 31/04; A61P 1/04

(52) U.S. Cl. CPC A61K 38/4886; A61P 1/00; C12Y 304/24087; A61P 1/04; A61P 31/04

(57) ABSTRACT

The methods and compositions described herein relate to the treatment of bowel inflammation, e.g., IBD or a thrombosis, ischemia, and/or pulmonaryembolism associated with IBD by administering ADAMTS13 to a subject.

### CROSS-REFERENCE TO RELATED APPLICATIONS

This application claims benefit under 35 U.S.C. § 119(e) of U.S. Provisional Application No. 62/182,087 filed Jun. 19, 2015, the contents of which are incorporated herein by reference in their entirety.

### GOVERNMENT SUPPORT

This invention was made with government support under Grant Nos. 1R01HL125501 awarded by the National Institutes of Health. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Jun. 10, 2016, is named 701039-085231-PCT_SL.txt and is 19,237 bytes in size.

### TECHNICAL FIELD

The technology described herein relates to the treatment of colitis and/or inflammatory bowel disease (IBD).

### BACKGROUND

Inflammatory bowel disease (IBD) is a disease of chronic intestinal inflammation manifesting in relapsing and remitting chronic often bloody diarrhea, debilitating abdominal pain, with significant morbidity and mortality. IBD affects 1.2 million individuals in the US with a rising incidence. The precise pathogenesis of IBD is unknown. It is thought to arise from a combination of genetic predisposition and a dysregulated immune response to a microbial trigger. Since there are a variety of genetic mutations and microbial triggers implicated in IBD, there are most likely multiple pathways that result in a similar phenotype of colitis. Patients with IBD are at three fold higher risk of developing thromboembolism compared to age matched controls, and this risk rises further to 15 fold with worsening disease activity. Deep vein thrombosis and pulmonary embolism are most common, but arterial events including increased incidence of cardiovascular disease and mesenteric ischemia are also reported (Ozturk et al. Inflammatory bowel diseases. 2015. doi: 10.1097/MIB.0000000000000355; Ha et al. The American journal of gastroenterology. 2009; 104(6):1445-51). These data suggest widespread endothelial activation and dysfunction in IBD. Since patients with IBD have multiple features of enhanced coagulation, platelet function abnormalities, thrombocytosis, as well as defects in fibrinolysis, some even postulate that thrombosis may play a role in IBD pathogenesis (Danese et al. The American journal of gastroenterology. 2007; 102(1):174-86; Harries et al. British medical journal. 1983; 286(6376):1476; Andoh et al. Journal of gastroenterology. 2006; 41(1):47-54). Increasing evidence links inflammation, and thrombosis in multiple chronic inflammatory states including IBD (Esmon. Thrombosis research. 2004; 114(5-6):321-7; Feys et al. British journal of haematology. 2007; 138(4):534-40).

Leukocyte rolling, adhesion and transmigration are hallmarks of inflammation. This process is mediated by several molecules on leukocytes and endothelial cells. In fact antibody blockade of adhesion molecules such as integrins, involved in trafficking of leukocytes to the gut are used to treat IBD (Sandborn et al. The New England journal of medicine. 2013; 369(8):711-21; Feagan et al. The New England journal of medicine. 2013; 369(8):699-710). Von Willebrand Factor (VWF), a large multimeric protein (ULVWF) stored in Weibel-Palade bodies of the endothelium, is released upon activation. VWF release leads to the initial adhesion of platelets and leukocytes, a first step in initiating inflammation and thrombosis (Wagner et al. Annual review of cell biology. 1990; 6:217-46; Ley et al. Nature reviews Immunology. 2007; 7(9):678-89). ULVWF multimers are extremely biologically active as they avidly bind to extracellular matrix and form stronger bonds with platelet GPIb compared to smaller plasma multimers. A disintegrin and metalloproteinase with thrombospondin type I repeats—motif 13 (ADAMTS13) decreases platelet adhesion and VWF platelet string formation by cleaving hyperactive ULVWF multimers under conditions of fluid shear stress (Dong et al. Blood. 2002; 100(12):4033-9). In fact mice completely deficient in ADAMTS13 (ADAMTS13−/−) have a pro-inflammatory and pro-thrombotic phenotype with increased: leukocyte rolling on unstimulated veins; leukocyte adhesion in inflamed venules; neutrophil influx in thioglycolate peritonitis; neutrophil recruitment into excisional skin wounds; and both plasma soluble p-selectin and VWF concentrations compared to WT (Chauhan et al. The Journal of experimental medicine. 2008; 205(9):2065-74).

Circulating VWF is elevated in IBD (Stevens et al. Gut. 1992; 33(4):502-6) and rises further with worsening colitis disease activity (Zezos et al. P. World journal of gastroenterology: WJG. 2005; 11(48):7639-45) reflecting activated endothelium with colonic inflammation. Furthermore, ADAMTS13 levels and activity are reduced in active IBD compared to levels in healthy control plasma, a phenomena observed in other diseases of chronic inflammation (Owczarek et al. Journal of Crohns and Colitis. 2015; 9(suppl 1):5208-9).

### SUMMARY

As described herein, subjects with decreased ADAMTS13 levels have more severe colitis, measured by increased weight loss, worse anemia clinical and histologic colitis severity. Decreases in ADAMTS13 also resulted in colitis cases with increased submucosal colonic thrombi. Administration of ADAMTS13 decreases colitis severity (reduced weight loss and clinical colitis scores), without worsening colonic bleeding, and reduced inflammatory molecules.

Accordingly, in one aspect, is a method of treating an inflammatory bowel disease (IBD), the method comprising administering ADAMTS13 to a subject in need thereof.

Another aspect provided herein relates to a method for treating a flare-up of an inflammatory bowel disease (IBD), the method comprising administering ADAMTS13 to a subject in need thereof.

Also provided herein, in another aspect, is a method of treating a symptom of an inflammatory bowel disease (IBD), the method comprising administering ADAMTS13 to a subject in need thereof.

In one embodiment of this aspect and all other aspects described herein, the symptom of inflammatory bowel disease (IBD) is inflammation of the colon or small intestine.

Another aspect provided herein relates to a method of preventing a thrombosis, ischemia, and/or pulmonary embolism associated with IBD, the method comprising administering ADAMTS13 to a subject in need thereof.

In one embodiment of this aspect and all other aspects described herein, the ADAMTS13 is administered intravenously.

In another embodiment of this aspect and all other aspects described herein, the ADAMTS13 is administered subcutaneously.

In another embodiment of this aspect and all other aspects described herein, the ADAMTS13 is a human ADAMTS13 polypeptide.

In another embodiment of this aspect and all other aspects described herein, the ADAMTS13 is recombinant ADAMTS13.

In another embodiment of this aspect and all other aspects described herein, the ADAMTS13 is plasma-derived ADAMTS13.

In another embodiment of this aspect and all other aspects described herein, the ADAMTS13 is administered at a dosage of from about 20 U/kg to about 1000 U/kg.

In another embodiment of this aspect and all other aspects described herein, the ADAMTS13 is administered at a dosage of from about 40 U/kg to about 500 U/kg.

In another embodiment of this aspect and all other aspects described herein, the ADAMTS13 is administered at a dosage of from about 40 U/kg to about 250 U/kg.

In another embodiment of this aspect and all other aspects described herein, the ADAMTS13 is administered at least every 2 days.

In another embodiment of this aspect and all other aspects described herein, the ADAMTS13 is administered daily.

In another embodiment of this aspect and all other aspects described herein, the method comprises (a) determining a level of ADAMTS13 expression in the subject; and (b) administering ADAMTS13 to the subject if the level of ADAMTS13 expression is reduced relative to the average level of ADAMTS13 expression in subjects without inflammatory bowel disease.

In another embodiment of this aspect and all other aspects described herein, the method comprises: (a) determining a level of ADAMTS13 activity in the subject; and (b) administering ADAMTS13 to the subject if the level of ADAMTS13 activity is reduced relative to the average level of ADAMTS13 activity in subjects without inflammatory bowel disease.

In another embodiment of this aspect and all other aspects described herein, the subject is administered ADAMTS13 in response to a flare-up of the IBD.

In another embodiment of this aspect and all other aspects described herein, the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.

In another embodiment of this aspect and all other aspects described herein, the idiopathic colitis is microscopic colitis, lymphocytic colitis, or collagenous colitis.

In another embodiment of this aspect and all other aspects described herein, the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis

In another embodiment of this aspect and all other aspects described herein, the infectious colitis is Clostridium difficile colitis.

Also provided herein in another aspect is a composition for use in the treatment of an inflammatory bowel disease (IBD), the composition comprising ADAMTS13.

Another aspect provided herein relates to a composition for use in treatment of a flare-up of an inflammatory bowel disease (IBD), the composition comprising ADAMTS13.

Also provided herein, in another aspect, is a composition for use in the treatment of a symptom of an inflammatory bowel disease (IBD), the composition comprising ADAMTS13.

In one embodiment of this aspect and all other aspects described herein, the symptom of inflammatory bowel disease (IBD) is inflammation of the colon or small intestine.

Another aspect provided herein relates to a composition for use in the prevention of a thrombosis, ischemia, and/or pulmonary embolism associated with IBD, the composition comprising ADAMTS13.

In one embodiment of this aspect and all other aspects described herein, the composition is formulated for intravenous administration.

In another embodiment of this aspect and all other aspects described herein, the composition is formulated for subcutaneous administration.

In another embodiment of this aspect and all other aspects described herein, the ADAMTS13 is a human ADAMTS13 polypeptide.

In another embodiment of this aspect and all other aspects described herein, the ADAMTS13 is recombinant ADAMTS13.

In another embodiment of this aspect and all other aspects described herein, the ADAMTS13 is plasma-derived ADAMTS13.

In another embodiment of this aspect and all other aspects described herein, the ADAMTS13 is formulated at a dosage of from about 20 U/kg to about 1000 U/kg.

In another embodiment of this aspect and all other aspects described herein, the ADAMTS13 is formulated at a dosage of from about 40 U/kg to about 500 U/kg.

In another embodiment of this aspect and all other aspects described herein, the ADAMTS13 is formulated at a dosage of from about 40 U/kg to about 250 U/kg.

In another embodiment of this aspect and all other aspects described herein, the ADAMTS13 is formulated for administration at least every 2 days.

In another embodiment of this aspect and all other aspects described herein, the ADAMTS13 is formulated for daily administration.

In another embodiment of this aspect and all other aspects described herein, the ADAMTS13 is formulated for administration in response to a flare-up of IBD.

In another embodiment of this aspect and all other aspects described herein, the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.

In another embodiment of this aspect and all other aspects described herein, the idiopathic colitis is microscopic colitis, lymphocytic colitis, or collagenous colitis.

In another embodiment of this aspect and all other aspects described herein, the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis

In another embodiment of this aspect and all other aspects described herein, the infectious colitis is Clostridium difficile colitis.

Also provided herein, in another aspect is the use of ADAMTS13 for the manufacture of a medicament for the treatment of inflammatory bowel disease (IBD).

Also provided herein, in another aspect is the use of ADAMTS13 for the manufacture of a medicament for the treatment of a flare-up of inflammatory bowel disease (IBD).

Also provided herein, in another aspect is the use of ADAMTS13 for the manufacture of a medicament for the treatment of a symptom of an inflammatory bowel disease.

In one embodiment of this aspect and all other aspects described herein, the symptom of inflammatory bowel disease (IBD) is inflammation of the colon or small intestine.

Also provided herein, in another aspect is the use of ADAMTS13 for the manufacture of a medicament for the prevention of a thrombosis, ischemia, and/or pulmonary embolism associated with IBD, the composition comprising ADAMTS13.

In one embodiment of this aspect and all other aspects described herein, the medicament is formulated for intravenous administration.

In another embodiment of this aspect and all other aspects described herein, the medicament is formulated for subcutaneous administration.

In another embodiment of this aspect and all other aspects described herein, the ADAMTS13 is a human ADAMTS13 polypeptide.

In another embodiment of this aspect and all other aspects described herein, the ADAMTS13 is recombinant ADAMTS13.

In another embodiment of this aspect and all other aspects described herein, the ADAMTS13 is plasma-derived ADAMTS13.

In another embodiment of this aspect and all other aspects described herein, the ADAMTS13 is formulated at a dosage of from about 20 U/kg to about 1000 U/kg.

In another embodiment of this aspect and all other aspects described herein, the ADAMTS13 is formulated at a dosage of from about 40 U/kg to about 500 U/kg.

In another embodiment of this aspect and all other aspects described herein, the ADAMTS13 is formulated at a dosage of from about 40 U/kg to about 250 U/kg.

In another embodiment of this aspect and all other aspects described herein, the ADAMTS13 is formulated for administration at least every 2 days.

In another embodiment of this aspect and all other aspects described herein, the ADAMTS13 is formulated for daily administration.

In another embodiment of this aspect and all other aspects described herein, the ADAMTS13 is formulated for administration in response to a flare-up of IBD.

In another embodiment of this aspect and all other aspects described herein, the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.

In another embodiment of this aspect and all other aspects described herein, the idiopathic colitis is microscopic colitis, lymphocytic colitis, or collagenous colitis.

In another embodiment of this aspect and all other aspects described herein, the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis

In another embodiment of this aspect and all other aspects described herein, the infectious colitis is Clostridium difficile colitis.

### BRIEF DESCRIPTION OF THE DRAWINGS

FIG. 1A depicts percent original weight loss in 17 WT and 15 ADAMTS13−/− mice. ADAMTS13−/− mice have significantly more weight loss on days 9-10 when their disease is most severe. FIG. 1B demonstrates that ADAMTS13−/− mice have a more severe colitis phenotype based on clinical colitis scores. Their disease onset tended to be earlier and was more severe days 8-10. FIG. 1C demonstrates that hemoglobin concentration in WT and ADAMTS13−/− mice without colitis is not significantly different at baseline. With colitis both WT and ADAMTS13−/− become anemic but the ADAMTS13−/− become more anemic suggesting more severe disease. FIG. 1D demonstrates that colon length is slightly shorter in ADAMTS13−/− mice than WT at baseline. With colitis ADAMTS13−/− mice have shorter colons than WT suggesting more edema and inflammation. FIG. 1E shows ADAMTS13−/− colons had more histologic evidence of inflammation compared to WT (FIG. 1E, p<0.05). FIG. 1F depicts histologic scoring of H+E stained colon sections from WT and ADAMTS13−/− mice, demonstrating more severe disease in ADAMTS13−/− mice. In all graphs mean and SEM are shown. * p<0.05, **p<0.005, ***p<0.0005. FIG. 1G shows circulating total leukocyte counts did not differ at baseline. However, with colitis ADAMTS13−/− mice had lower total circulating leukocytes compared to WT. FIG. 1H shows neutrophils increased in WT mice with colitis but were unchanged in ADAMTS13−/− mice with colitis. FIG. 1I shows lymphocytes in WT mice were unchanged, with colitis and tended to be lower in ADAMTS13−/− mice compared to WT. FIG. 1J shows monocytes rose significantly in both WT and ADAMTS13−/− mice with colitis but did not differ between the two genotypes.

FIG. 2A depicts graphs demonstrating that platelet counts for WT and ADAMTS13−/− mice without colitis do not differ. With colitis there is significant thrombocytosis in WT mice while ADAMTS13−/− mice vary more widely but have a similar mean to baseline. FIG. 2B depicts a graph of colons from WT and ADAMTS13−/− mice fluorescently stained for VWF, PECAM and DNA. Entire examined Swiss rolled sections of colon were inspected for thrombi. One or more VWF rich thrombi were found in ADAMTS13−/− colons and none in WT colons with colitis. In both graphs mean and SEM are shown. * p<0.05, **p<0.005, ^{***}p<0.0005. In WT we see increased VWF expression mostly within the vessel wall with likely some VWF staining platelets in the lumen. In contrast in ADAMTS13−/− mice a completely occlusive VWF rich thrombus is found in a submucosal vessel.

FIG. 3A depicts a graph demonstrating that both WT and ADAMTS13−/− had increased circulating VWF with colitis. The plot displays the fold increase over genotype mean without and with DSS treatment. (**p<0.005, ***p<0.0005). FIG. 3B depicts a graph in which, using Image J software VWF area over DNA area (to represent total tissue present) was quantified. Significantly more VWF positive staining was found in both WT and ADAMTS13−/− with colitis. There was a trend towards more VWF in ADAMTS13−/− mice compared to WT. VWF was then quantified at severely affected ulcerated areas on another experimental group (to avoid counting the same areas twice) and even higher VWF staining was found with increased tissue injury. Mean and SEM are shown.

FIG. 4A depicts a graph of percent of original body weight in mice with DSS (squares) and rhADAMTS13 (circles) or vehicle treatment. There is significantly less weight loss in rhADAMTS13−/− treated mice on days 7-10 when colitis is most severe. FIG. 4B depicts a graph of colitis scores, which are more severe on days 8 and 9 in vehicle treated compared rhADAMTS13 treated mice. FIG. 4C depicts a graph demonstrating that hemoglobin decreases with colitis in both vehicle and rhADAMTS13 treated mice. However, there is no significant difference in hemoglobin concentration between either treatment group. In both graphs mean and SEM are shown. * p<0.05, **p<0.005, ***p<0.0005. FIG. 4D depicts a platelet graph showing less thrombocytosis in the rhADAMTS13 treated group. FIG. 4E depicts a graph demonstrating that plasma IL-6, a pro-inflammatory and prothrombotic cytokine was lower in the sera of rhADAMTS13−/− mice compared to vehicle treated colitic mice.

FIG. 5A No significant difference was seen in plasma IL-6 in WT or ADAMTS13−/− mice with colitis. There were no differences in plasma IL-10 (FIG. 5B), sPsel (FIG. 5C) or TAT (FIG. 5D) between WT mice with DSS or ADAMTS13−/− with DSS or between mice and WT mice treated with rhADAMTS13 or vehicle.

### DETAILED DESCRIPTION

As described herein, decreased activity and/or levels of ADAMTS13 can contribute to symptoms associated with inflammatory conditions of the colon and small intestines. Surprisingly, it was found that ADAMTS13 deficient mice did not become thrombocytotic upon chemical induction of acute colitis. This led to the discovery of increased ultra large VWF (ULVWF) release upon acute colonic injury, and localization to ulcerated sites. Advantageously, it is shown herein that administration of ADAMTS13 reduced symptoms associated with chemically induced acute colitis. For example, it is reported in FIG. 6 that administration of recombinant ADAMTS13 reduced weight loss and improved the colitis score of mice with acute DSS-induced colitis (p<0.05).

Anemia is a common symptom associated with IBD (Antunes C. V., et al., Biomed Res. Int., 2015:728925 (2015)), presumably due to increased gastrointestinal bleeding and reduced absorption of nutrients required for red blood cell replenishment. Because ADAMTS13 functions to cleave VWF into smaller molecules, which are less competent for blood coagulation, it may be predicted that administration of recombinant ADAMTS13 would further increase gastrointestinal bleeding by inhibiting blood clot formation. Unexpectedly, administration of ADAMTS13 did not increase anemia in the acute colitis mouse model, despite active colonic bleeding and administration of ADAMTS13 doses many times greater than native ADAMTS13 levels in mice.

Thus, in one aspect, described herein is a method of treating colitis in a subject in need of treatment thereof, the method comprising administering ADAMTS13 or an ADAMTS13 agonist to the subject. In some embodiments, the colitis is associated with inflammatory bowel disease (IBD). In one aspect, described herein is a method of treating inflammatory bowel disease (IBD) in need of treatment thereof, the method comprising administering ADAMTS13 or an ADAMTS13 agonist to the subject. In one aspect, described herein is a method of preventing and/or reducing thrombosis, ischemia, and/or pulmonary embolism in a subject having IBD, the method comprising administering ADAMTS13 or an ADAMTS13 agonist to the subject.

Furthermore, rhADAMTS13 can be of therapeutic benefit in decreasing complications of chronic systemic inflammation associated with IBD such as increased risk of stroke and cardiovascular disease, as well as deep vein thrombosis (DVT) and pulmonary embolism (PE). Since rhADAMTS13 decreased platelet/leukocyte extravasation, and blood vessel permeability in addition to its ability to decrease thrombotic risk it also demonstrates an anti-inflammatory effect. Currently anti-leukocyte adhesion therapies are used to treat IBD. rhADAMTS13 may have similar anti-inflammatory effects when used alone or in combination with other anti-adhesion molecules to treat IBD.

### Definitions

As used herein, the terms “colitides,” “inflammatory bowel disease,” and “IBD” refer to inflammatory conditions of the colon and/or small intestine, often characterized by abdominal pain, vomiting, diarrhea, rectal bleeding, cramps, and/or anemia. Examples of IBD include Crohn's disease, ulcerative colitis, and various classifications of colitides, e.g., idiopathic colitides (e.g., microscopic colitis, lymphocytic colitis, and collagenous colitis), iatrogenic colitides (e.g., diversion colitis, neutropenic enterocolitis, disinfectant colitis, corrosive colitis, nonsteroidal anti-inflammatory drug and salicylate-induced colitis, toxic epidermal necrolysis, and other chemical-induced colitides), ischemic colitis, infectious colitides (e.g., Clostridium difficile colitis), eosinophilic colitis. In some embodiments, the condition (e.g., the IBD) is chronic, acute, and/or recurring.

As used herein, a “flare” or “flare-up” of an inflammatory bowel disease (e.g., a colitis or Crohn's disease) refers to an acute aggravation of a symptom associated with the inflammatory bowel disease. Non-limiting examples of symptoms that, when increased in magnitude, are indicative of an IBD flare-up include, abdominal pain (e.g., pain that is unresponsive or less responsive to conventional management with, for example, pain medication or anti-spasmodic agents), increased rectal bleeding or blood in the subject's stool, an increase in the frequency of loose bowel movements, loss of appetite, and increased abdominal inflammation.

“ADAMTS13” or “a disintegrin and metalloproteinase with a thrombospondin type 1 motif, member 13” is a zinc-containing metalloprotease that cleaves von Willebrand factor (VWF), degrading large VWF multimers in the blood and thereby inhibiting blood clotting. ADAMTS13 is also known in the art as von Willebrand factor-cleaving protease (VWFCP). As used herein, “ADAMTS13” or “A13” refers to any protein or polypeptide with ADAMTS13 activity, particularly the ability to cleave the peptide bond between residues Tyr-842 and Met-843 of human VWF. In an exemplary embodiment, an ADAMTS13 protein refers to a polypeptide comprising an amino acid sequence that has high sequence identity (e.g., at least 70%, 75%, 80%, 85%, 90%, 95%, or more) to a wildtype ADAMTS13 protein. Non-limiting examples of wildtype ADAMTS13 proteins include NP_620594 (HsADAMTS13 isoform 1, preproprotein) or amino acids 75 to 1427 of NP 620594 (HsADAMTS13 isoform 1, mature polypeptide), NP 620596 (HsADAMTS13 isoform 2, preproprotein) or amino acids 75 to 1371 of NP_620594 (HsADAMTS13 isoform 2, mature polypeptide), and NP 620595 (HsADAMTS13 isoform 3, preproprotein) or amino acids 75 to 1340 of NP 620595 (HsADAMTS13 isoform 1, mature polypeptide). As used herein, an ADAMTS13 protein includes natural variants with VWF cleaving activity and artificial constructs with VWF cleaving activity. As used in the present disclosure, ADAMTS13 encompasses any natural variants, alternative sequences, isoforms or mutant proteins that retain some basal activity. Examples of ADAMTS13 amino acid variations (relative to SEQ ID NO:2) found in the human population include, without limitation, R7W, I79M, V88M, H96D, R102C, S119F, I178T, R193W, T1961, 5203P, L232Q, H234Q, D235H, A250V, S263C, R268P, Y304C, C311Y, C347S, R349C, P353L, W390C, R398H, Q448E (rs2301612), Q456H, P457L, R507Q, C508Y, G525D, R528G, A606P, P618A (rs28647808), R625H, Y658C, P671L, I673F, R692C, A732V (rs41314453), C758R, S903L, C908S, C908Y, C951G, G982R (rs36222275), C1024G, A1033T, R1060W (rs142572218), R1095W, R1123C, C1213Y, R1219W, T1226I, G1239V, R1336W, many of which have been found associated with thrombotic thrombocytopenic purpura (TTP). ADAMTS13 variants that have been shown to be associated with ADAMTS13 activity include A732V or rs41314453, rs3118667, D187H or rs148312697, D187A, R1060W or rs142572218, and G982R or rs36222275, and are described in “Genetic variants in the ADAMTS13 and SUPT3H genes are associated with ADAMTS13 activity,” Paul S. de Vries et al., Jun. 18, 2015; Blood: 125 (25), the contents of which are herein incorporated by reference in their entireties. ADAMTS13 proteins also include polypeptides containing post-translational modifications. For example, ADAMTS13 has been shown to be modified by N-acetylglucosamine (GlcNAc) at residues 614, 667, and 1354, and it has been predicted that residues 142, 146, 552, 579, 707, 828, and 1235 may also be modified in this fashion.

In some embodiments, an ADAMTS13 polypeptide includes the human polypeptide (e.g., SEQ ID NO: 2); as well as homologs from other species, including but not limited to bovine, dog, cat chicken, murine, rat, porcine, ovine, turkey, horse, fish, baboon and other primates. The terms also refer to fragments or variants of the native polypeptide that maintain at least 50% of the activity or effect of the native full length polypeptide, e.g. as measured in an appropriate animal model. Conservative substitution variants that maintain the activity of wildtype polypeptides will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants can be tested for activity, for example, by administering the variant to an appropriate animal model of acute colitis, e.g., a DSS-induced colitis mouse model, as described herein. Variants can also be tested for activity, for example, by use of a suitable in vitro VWF cleavage assay, e.g., using a FRET-VWF73 substrate (Kokame et al., Br J Haematol. 2005 April; 129(1):93-100).

In some embodiments, a polypeptide, e.g., an ADAMTS13 polypeptide, can be a variant of a sequence described herein, e.g. a variant of an ADAMTS13 polypeptide comprising the amino acid sequence of SEQ ID NO: 2. In some embodiments, the variant is a conservative substitution variant. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wildtype, e.g. 110%, 125%, 150%, 175%, 200%, 500%, 1000% or more.

As used herein, an agonist refers to any agent that increases the level and/or activity of the target, e.g., of ADAMTS13. As used herein, the term “agonist” refers to an agent which increases the expression and/or activity of the target by at least 10% or more, e.g. by 10% or more, 50% or more, 100% or more, 200% or more, 500% or more, or 1000% or more. The efficacy of an agonist of, for example, ADAMTS13, e.g. its ability to increase the level and/or activity of ADAMTS13 can be determined, e.g. by measuring the level of an expression product of ADAMTS13 and/or the activity of ADAMTS13. Methods for measuring the level of a given mRNA and/or polypeptide are known to one of skill in the art, e.g. RT-PCR with primers can be used to determine the level of RNA, and Western blotting with an antibody (e.g. an anti-ADAMTS13 antibody, e.g. Cat No. ab90786 Abcam; Cambridge, Mass.) can be used to determine the level of a polypeptide. The activity of, e.g. ADAMTS13 can be determined using methods known in the art see, e.g., Crist and Rogers. Lab Medicine 2009 40:232-235; which is incorporated by reference herein in its entirety, for descriptions of commercial and non-commercial ADAMTS13 activity assays. Commercially available assays can include ADAMTS-13 Activity Assay (GTI, Waukesha, Wis.) and the Technozym ADAMTS-13 ELISA kit (Technoclone, Vienna, Austria).

Non-limiting examples of agonists of ADAMTS13 include nucleic acids encoding a ADAMTS13 polypeptide, e.g. a polypeptide comprising the sequence SEQ ID NO: 2 or a nucleic acid comprising the sequence of SEQ ID NO: 1 or variants thereof.

In some embodiments of any of the aspects described herein, the subject is a subject having a condition or diagnosed as having colitis or IBD. In some embodiments, the subject is a human diagnosed with a subtype of IBD (e.g., Crohn's disease, ulcerative colitis, or any other colitis described herein.

In some embodiments of any of the aspects described herein, the subject is a subject at risk or at increased risk of developing colitis or IBD (e.g., a human subject). In some embodiments of any of the aspects described herein, the subject can be a subject with colitis or IBD at risk or at increased risk of developing thrombosis, embolism, pulmonary embolism, or ischemia. In some embodiments, described herein is a method of treating or preventing thrombosis, embolism, pulmonary embolism, or ischemia in a subject having or diagnosed as having colitis or IBD. In some embodiments of any of the aspects described herein, the subject at risk or at increased risk of developing thrombosis, embolism, pulmonary embolism, or ischemia can be a subject having a condition or diagnosed as having colitis or IBD. In some embodiments of any of the aspects described herein, the subject can be a subject determined to have a reduced level of expression and/or activity of ADAMTS13.

In some embodiments of any of the aspects described herein, the subject has a reduced level of expression and/or activity of ADAMTS13. In some embodiments, a subject with a reduced level of expression of ADAMTS13 is a subject with a level of expression of ADAMTS13 that is 75% or less of a reference level, e.g., 75% or less, 70% or less, 60% or less, 50% or less, 40% or less, 30% or less, 20% or less, or 10% or less. In some embodiments, the reference level of ADAMTS13 expression is an average physical concentration of ADAMTS13 protein in the plasma (e.g., protein per mL) of a representative population. For example, in some embodiments, the reference is an average physical concentration of ADAMTS13 in the plasma of at least 100 subjects without IBD.

In some embodiments, a subject with a reduced level of ADAMTS13 activity is a subject with a level of ADAMTS13 activity that is 75% or less of a reference level, e.g., 75% or less, 70% or less, 60% or less, 50% or less, 40% or less, 30% or less, 20% or less, or 10% or less. In some embodiments, the reference level of ADAMTS13 activity is an average amount of ADAMTS13 activity (e.g., units per mL) in the plasma of a representative population. For example, in some embodiments, the reference is an average amount of ADAMTS13 activity in the plasma of at least 100 subjects without IBD.

In some embodiments of any of the aspects described herein, a subject can be determined to have a reduced level of expression and/or activity of ADAMTS13, in part, by determining whether the subject has one or more genetic variations known to be associated with reduced expression and/or activity of ADAMTS13, such as one or more of A732V (rs41314453), D187H (rs148312697), D187A, R1060W (rs142572218), G982R (rs36222275), and P618A (rs28647808).

The term “sample” or “test sample” as used herein denotes a sample taken or isolated from a biological organism, e.g., a blood or plasma sample from a subject. Exemplary biological samples include, but are not limited to, a biofluid sample; serum; plasma; urine; saliva; and/or tissue sample etc. The term also includes a mixture of the above-mentioned samples. The term “test sample” also includes untreated or pretreated (or pre-processed) biological samples. In some embodiments, a test sample can comprise cells from a subject. In some embodiments, the test sample can be blood; plasma; urine, or serum.

The test sample can be obtained by removing a sample from a subject, but can also be accomplished by using a previously isolated sample (e.g. isolated at a prior timepoint and isolated by the same or another person).

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject. In some embodiments, the subject can be a subject in need of treatment for (e.g. having or diagnosed as having) colitis, IBD, thrombosis or a subject at risk of or at increased risk of developing a colitis, IBD, or thrombosis as described elsewhere herein. In some embodiments, the subject can be a subject determined to have one or more genetic variations known to be associated with reduced expression and/or activity of ADAMTS13, such as one or more of A732V (rs41314453), D187H (rs148312697), D187A, R1060W (rs142572218), G982R (rs36222275), and P618A (rs28647808).

In some embodiments, the methods described herein relate to treating a subject having or diagnosed as having colitis with ADAMTS13 or an agonist of ADAMTS13. Subjects having colitis can be identified by a physician using current methods of diagnosing colitis. Symptoms and/or complications of colitis which characterize these conditions and aid in diagnosis are well known in the art and include but are not limited to, abdominal pain, loss of appetite, fatigue, bloody diarrhea, mucus in the stool, cramping, weight loss, and fever. Tests that may aid in a diagnosis of, e.g. colitis include, but are not limited to, stool culture, abdominal CT or x-ray, or colonoscopy. A family history of colitis, or exposure to risk factors for colitis can also aid in determining if a subject is likely to have colitis or in making a diagnosis of colitis.

The compositions and methods described herein can be administered to a subject having or diagnosed as having a condition described herein, e.g. colitis or IBD. In some embodiments, the methods described herein comprise administering an effective amount of compositions described herein, e.g. ADAMTS13 or an agonist of ADAMTS13 to a subject in order to alleviate a symptom of a condition. As used herein, “alleviating a symptom” is ameliorating any condition or symptom associated with a condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of, e.g., an agonist of ADAMTS13 needed to alleviate at least one or more symptom of the disease or disorder, and relates to a sufficient amount of pharmacological composition to provide the desired effect. The term “therapeutically effective amount” therefore refers to an amount of a compound that is sufficient to provide a particular therapeutic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for ADAMTS13 levels among others. The dosage can be determined by a physician and adjusted, as necessary, to suit observed effects of the treatment.

In some embodiments, the technology described herein relates to a pharmaceutical composition comprising an agonist of ADAMTS13 as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” or the like are used interchangeably herein. In some embodiments, the carrier inhibits the degradation of the active agent, e.g. an agonist of ADAMTS13 as described herein.

In some embodiments, the pharmaceutical composition comprising ADAMTS13 or an agonist of ADAMTS13 as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of ADAMTS13 or an agonist of ADAMTS13 as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a compound as disclosed herein can also be incorporated into the parenteral dosage forms of the disclosure, including conventional and controlled-release parenteral dosage forms.

Pharmaceutical compositions comprising ADAMTS13 or an agonist of ADAMTS13 can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the ADAMTS13 or an agonist of ADAMTS13 can be administered in a sustained release formulation.

The efficacy of ADAMTS13 or an agonist of ADAMTS13 in, e.g., the treatment of a condition described herein, or to induce a response as described herein (e.g. an increase in ADAMTS13 expression and/or activity levels) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. ADAMTS13 levels. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. symptoms of colitis and/or ADAMTS13 levels). It is well within the ability of one skilled in the art to monitor efficacy of administration and/or treatment by measuring any one of such parameters, or any combination of parameters. Efficacy can be assessed in animal models of a condition described herein, for example treatment of mouse models of colitis. When using an experimental animal model, efficacy of treatment is evidenced when a statistically significant change in a marker is observed, e.g. ADAMTS13 levels.

In vitro and animal model assays are provided herein which allow the assessment of a given dose of ADAMTS13 or an agonist of ADAMTS13. By way of non-limiting example, the effects of a dose of an agonist of ADAMTS13 can be assessed by administering the ADAMTS13 or agonist to ADAMTS13−/− mice, e.g. with or without secondary colitis-inducing factors. The agonist can be administered, e.g., by intravenous injection. Efficacy can be measured by, e.g., inflammation, IL-6 levels, percent weight loss, colitis scores, or VWF levels.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease,” “reduced,” “reduction,” or “inhibit” are all used herein to mean a decrease by a statistically significant amount. In some embodiments, “reduce,” “reduction,” “decrease,” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “enhance,” or “activate” are all used herein to mean an increase by a statically significant amount. In some embodiments, the terms “increased,” “increase,” “enhance,” or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. In some embodiments, the subject is a mammal, e.g., a primate, e.g., a human. The terms, “individual,” “patient,” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of, e.g., colitis. A subject can be male or female.

A subject can be one who has been previously diagnosed with or identified as suffering from or having a condition in need of treatment (e.g. colitis) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, “engineered” refers to the aspect of having been manipulated by the hand of man. For example, an ADAMTS13 polypeptide is considered to be “engineered” when the sequence of the polypeptide and/or encoding nucleic acid sequence manipulated by the hand of man to differ from the sequence of an polypeptide as it exists in nature. As is common practice and is understood by those in the art, progeny and copies of an engineered polynucleotide and/or polypeptide are typically still referred to as “engineered” even though the actual manipulation was performed on a prior entity.

As used herein, “recombinant” refers to a cell, tissue or organism that has undergone transformation with a new combination of genes or DNA. When used in reference to nucleic acid molecules, “recombinant” refers to a combination of nucleic acid molecules that are joined together using recombinant DNA technology into a progeny nucleic acid molecule, and/or a heterologous nucleic acid sequence introduced into a cell, tissue, or organism. When used in reference to a polypeptide, “recombinant” refers to a polypeptide which is the expression product of a recombinant nucleic acid, and can be such a polypeptide as produced by a recombinant cell, tissue, or organisms. The nucleic acid molecule can be stably integrated into the genome of the host or the nucleic acid molecule can also be present as an extrachromosomal molecule. Such an extrachromosomal molecule can be auto-replicating. Recombinant viruses, cells, and organisms are understood to encompass not only the end product of a transformation process, but also recombinant progeny thereof.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

One method of identifying amino acid residues which can be substituted is to align, for example, the human polypeptide to a homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g. SEQ ID NO: 2 or a nucleic acid encoding that amino acid sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, a polypeptide, e.g., an ADAMTS13 polypeptide, administered to a subject can comprise one or more amino acid substitutions or modifications. In some embodiments, the substitutions and/or modifications can prevent or reduce proteolytic degradation and/or prolong half-life of the polypeptide in the subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., an ADAMTS13 polypeptide, as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., an ADAMTS13 polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, an ADAMTS13 polypeptide as described herein can comprise alternative amino acids. Non-limiting examples of alternative amino acids include D-amino acids, beta-amino acids, homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. an ADAMTS13 polypeptide, can be modified, e.g. by addition of a moiety to one or more of the amino acids comprising the peptide. In some embodiments, a polypeptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG or albumin.

In some embodiments, the polypeptide administered to the subject (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative or non-conservative substitutions of the sequences disclosed herein.

Alterations of the original amino acid sequence can be accomplished by any of a number of techniques known to one of skill in the art. Amino acid substitutions can be introduced, for example, at particular locations by synthesizing oligonucleotides containing a codon change in the nucleotide sequence encoding the amino acid to be changed, flanked by restriction sites permitting ligation to fragments of the original sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, a polypeptide as described herein can be chemically synthesized and mutations can be incorporated as part of the chemical synthesis process.

Proteolytically active recombinant ADAMTS13 may be prepared by expression in mammalian cell cultures, as described in Plaimauer et al., (2002, Blood. 15; 100(10):3626-32), U.S. Patent Publication Nos. 2005/0266528 and 2012/0034674, and U.S. Pat. No. 8,313,926, the disclosures of which are herein incorporated by reference in their entireties for all purposes. Methods of recombinant culture of ADAMTS13 expressing cells are disclosed in Plaimauer B, Scheiflinger F. (Semin Hematol. 2004 January; 41(1):24-33, the disclosure of which is herein incorporated by reference in its entirety for all purposes).

As used herein, “one unit of ADAMTS activity” is defined as the amount of activity in 1 mL of pooled normal human plasma, regardless of the assay being used. For example, when the ADAMTS protein is ADAMTS13, one unit of ADAMTS13 FRETS-VWF73 activity is the amount of activity needed to cleave the same amount of FRETS-VWF73 substrate (Kokame et al., Br J Haematol. 2005 April; 129(1):93-100) as is cleaved by one mL of pooled normal human plasma. Conveniently, ADAMTS13 activity may be determined by functional assays, such as functional assays employing modified von Willebrand factor peptides as substrate for ADAMTS13 (Tripodi et al. J Thromb Haemost. 2008 September; 6(9): 1534-41). In one embodiment, to be considered as a ADAMTS13 protein as defined above, a polypeptide or protein must have at least 1% of the VWF cleaving activity of native ADAMTS13. In other embodiments, an ADAMTS13 protein will contain at least 10% of the activity of native ADAMTS13. In yet other embodiments, an ADAMTS13 protein will contain at least 5%, 10%, 20%, 30%, 40%, 50%, 60%, 70%, 80%, 90%, or 100% of the activity of native ADAMTS13. The quantity of an ADAMTS13 protein may also be determined by measurement of an ADAMTS13 antigen, for example using the ELISA method disclosed in Rieger et al., (2006, Thromb Haemost. 2006 95(2):212-20).

In some embodiments, the technology described herein relates to a nucleic acid encoding a polypeptide (e.g. an ADAMTS13 polypeptide) as described herein. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the nucleic acid is DNA. In another aspect, the nucleic acid is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

In some embodiments, a nucleic acid encoding a polypeptide as described herein (e.g. an ADAMTS13 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, “expression level” refers to the number of mRNA molecules and/or polypeptide molecules encoded by a given gene that are present in a cell or sample. Expression levels can be increased or decreased relative to a reference level.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. colitis. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” and all grammatical equivalents, refers to the placement of a compound as disclosed herein into a subject by a method or route which results in at least partial delivery of the agent at a desired site. Administration includes intravenous administration, intramuscular administration, subcutaneous administration, oral administration, administration as a suppository, topical contact, intraperitoneal, intralesional, or intranasal administration, or the implantation of a slow-release device, e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

### Exemplary Embodiments

The present disclosure is based, in part, on the surprising discovery that administration of ADAMTS13 alleviated symptoms of inflammatory bowel disease (IBD) in a mouse model of acute colitis. Equally surprising was the finding that, despite administration of ADAMTS13 activity doses that were several orders of magnitude greater than native activity in mice, ADAMTS13 administration did not increase anemia in the mouse model, which displayed active colonic bleeding.

Thus, in some embodiments, methods are provided for treating IBD (e.g., for managing a symptom associated with IBD) by administering ADAMTS13 to a subject in need thereof (e.g., a subject experiencing a flare-up of IBD).

In some embodiments of the methods described herein, the ADAMTS13 administered to the subject is recombinantly expressed. In other embodiments of the methods provided herein, the ADAMTS13 administered to the subject is plasma-derived (e.g., enriched from plasma, e.g., pooled human plasma). As described herein, the ADAMTS13 compositions used in the methods may include one or more different ADAMTS13 variants (including full-length and truncated forms). In some embodiments, the ADAMTS13 polypeptides have at least 70%, 75%, 80%, 85%, 90%, 91%, 92%, 93%, 94%, 95%, 96%, 97%, 98%, 99% and 100% sequence identity to any of the ADAMTS13 sequences described herein (e.g., any pre- or post-processed ADAMTS13 isoform or natural variant described above).

In some embodiments, the methods described herein include administration of ADAMTS13 (or an ADAMTS13 agonist) to a subject for treatment of a flare-up of IBD. In some embodiments, the methods include administration of ADAMTS13 (or an ADAMTS13 agonist) to a subject for treatment or management of a symptom of a flare-up of IBD (e.g., abdominal pain, rectal bleeding or blood in the subject's stool, loose bowel movements, loss of appetite, or abdominal inflammation).

In some embodiments, the methods described herein include administration of ADAMTS13 (or an ADAMTS13 agonist) to a subject for the treatment of IBD. In some embodiments, the methods described herein include administration of ADAMTS13 (or an agonist of ADAMTS13) for treatment or management of a symptom of IBD (e.g., abdominal pain, rectal bleeding or blood in the subject's stool, loose bowel movements, loss of appetite, or abdominal inflammation).

In some embodiments, the methods described herein include administration of ADAMTS13 (or an ADAMTS13 agonist) to a subject for the prophylactic treatment of IBD. In some embodiments, the methods described herein include administration of ADAMTS13 (or an agonist of ADAMTS13) to a subject with an increased risk of developing IBD (e.g., an increased familial risk, an increased genetic risk, or an increased environmental risk of developing one or more symptoms of IBD).

In some embodiments, the method includes administration of an ADAMTS13 agonist (e.g., an ADAMTS13 polypeptide) to a subject who meets particular clinical criteria associated with an ineffective treatment or flare-up of an IBD. E.g., in some embodiments, ADAMTS13 (or an ADAMTS13 agonist) is administered to a subject found to have a particular clinical criterion associated with IBD. In some embodiments, administration of ADAMTS13 is repeated until the subject falls below a particular criterion associated with IBD. In some embodiments, identification of the clinical criteria includes determination of a disease activity index for the particular subtype of inflammatory bowel disease.

In some embodiments, the Crohn's Disease Activity Index (CDAI) is used to identify a clinical criterion associated with Crohn's disease. In some embodiments, the Harvey-Bradshaw Index (HBI) is used to identify a clinical criterion associated with Crohn's disease. In some embodiments, ADAMTS13 is administered to a subject identified with an HBI score of at least 4. In some embodiments, ADAMTS13 is administered to a subject identified with an HBI score of at least 5. In some embodiments, ADAMTS13 is administered to a subject with a CDAI or HBI score that is higher than a baseline CDAI or HBI score for the patient, indicative of a remission or flare-up of the Crohn's disease (e.g., an HBI score that is at least 1 or 2 points higher than a baseline score for the subject).

In some embodiments, a Mayo score is used to identify a clinical criterion associated with ulcerative colitis. In some embodiments, a partial Mayo score is used to identify a clinical criterion associated with ulcerative colitis. In some embodiments, ADAMTS13 is administered to a subject with a Mayo score or partial May score that is higher than a baseline Mayo or partial Mayo, score for the subject (e.g., a Mayo score that is at least 1.5, 2.0, or 2.5 points higher than a baseline Mayo score for the subject; or a partial Mayo score that is at least 0.5, 1.0, or 1.5 points higher than a baseline partial Mayo score for the subject).

In some embodiments, a pediatric ulcerative colitis activity index (PUCAI) is used to identify a clinical criterion associated with ulcerative colitis in pediatric subjects. In some embodiments, ADAMTS13 is administered to a pediatric subject identified with a PUCAI score of at least about 35, 40, 45, 50, 55, 60, 65, 70, or 75. In some embodiments, ADAMTS13 is administered to a pediatric subject with a PUCAI score that is higher than a baseline PUCAI score for the pediatric subject (e.g., a PUCAI score that is at least 5, 10, or 15 points higher than a baseline PUCAI score for the pediatric subject).

In some embodiments, a measure of infection is used to identify a clinical criterion associated with an infectious colitides (e.g., Clostridium difficile colitis). In some embodiments, a total viable count (e.g., in the stool of the subject or in a sample from the bowel or intestine of the subject) of an infectious agent associated with the infectious colitis (e.g., Clostridium difficile) is the clinical criterion associated with the colitis. In some embodiments, a total spore count (e.g., in the stool of the subject or in a sample from the bowel or intestine of the subject) of an infectious agent associated with the infectious colitis (e.g., Clostridium difficile) is the clinical criterion associated with the colitis. In some embodiments, another marker of the presence of the infectious agent associated with the infectious colitis (e.g., a protein or nucleic acid specific to the infectious agent) is the clinical criterion associated with the colitis.

In some embodiments, ADAMTS13 (or an ADAMTS13 agonist) is administered as a second line therapy. For example, in some embodiments, ADAMTS13 (or an ADAMTS13 agonist) is administered to a subject who is partially or completely unresponsive to a first-line IBD treatment (e.g., an anti-inflammatory or immunosuppressant agent). In some embodiments, ADAMTS13 (or an ADAMTS13 agonist) is administered to a subject with a decreased chance of responding to a conventional IBD therapy (e.g., an anti-inflammatory or immunosuppressant agent).

Administration and Dosing

In some embodiments, the ADAMTS13 (or ADAMTS13 agonist) compositions disclosed herein are administered to the recipient by intravenous, subcutaneous, and/or intramuscular means. In some embodiments, ADAMTS13 (or an ADAMTS13 agonist) is administered systemically to the subject. Systemic administration includes without limitation oral, transdermal, subdermal, intraperitoneal, intravenous, subcutaneous, transnasal, sublingual, or rectal. In some embodiments, ADAMTS13 (or an ADAMTS13 agonist) is administered locally to the subject. Local administration includes, without limitation, administration directly to the colon or intestine via perfusion, injection, or other contact with the colon or intestine, or delivery via a sustained delivery device implanted at or near the colon or intestine.

In one embodiment, the method includes intravenous administration of ADAMTS13 (or an ADAMTS13 agonist). In one embodiment, the method includes subcutaneous administration of ADAMTS13 (or an ADAMTS13 agonist)

In some embodiments, an ADAMTS13 pharmaceutical composition is administered to the subject at a dose of about 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 1600, 1750, 2000, 2500, 3000, 3500, 4000, 5000, 6000, 7000, 8000, 9000, or 10,000 U A13/kg body weight of the subject. In some embodiments, the ADAMTS13 composition is administered to the subject at a dose of from 20 U/kg to 4000 U/kg body weight of the subject. In some embodiments, the ADAMTS13 composition is administered to the subject at a dose of from 20 U/kg to 1000 U/kg body weight of the subject. In some embodiments, the ADAMTS13 composition is administered to the subject at a dose of from 40 U/kg to 500 U/kg body weight of the subject. In some embodiments, the ADAMTS13 composition is administered to the subject at a dose of from 100 U/kg to 250 U/kg body weight of the subject. In some embodiments, the ADAMTS13 composition is administered to the subject at a dose of from 20 U/kg to 500 U/kg body weight of the subject. In some embodiments, the ADAMTS13 composition is administered to the subject at a dose of from 20 U/kg to 250 U/kg body weight of the subject. In some embodiments, the ADAMTS13 composition is administered to the subject at a dose of from 20 U/kg to 100 U/kg body weight of the subject. In some embodiments, the ADAMTS13 composition is administered to the subject at a dose of from 40 U/kg to 250 U/kg body weight of the subject. In some embodiments, the ADAMTS13 composition is administered to the subject at a dose of from 40 U/kg to 100 U/kg body weight of the subject. In some embodiments, the ADAMTS13 composition is administered to the subject at a dose of from 100 U/kg to 1000 U/kg body weight of the subject. In some embodiments, the ADAMTS13 composition is administered to the subject at a dose of from 100 U/kg to 500 U/kg body weight of the subject. In some embodiments, the ADAMTS13 composition is administered to the subject at a dose of from 100 U/kg to 250 U/kg body weight of the subject. In some embodiments, the ADAMTS13 composition is administered to the subject at a dose of from 250 U/kg to 2000 U/kg body weight of the subject. In some embodiments, the ADAMTS13 composition is administered to the subject at a dose of from 250 U/kg to 1000 U/kg body weight of the subject. In some embodiments, the ADAMTS13 composition is administered to the subject at a dose of from 250 U/kg to 500 U/kg body weight of the subject. In some embodiments, the ADAMTS13 composition is administered to the subject at a dose of from 500 U/kg to 4000 U/kg body weight of the subject. In some embodiments, the ADAMTS13 composition is administered to the subject at a dose of from 500 U/kg to 2000 U/kg body weight of the subject. In some embodiments, the ADAMTS13 composition is administered to the subject at a dose of from 500 U/kg to 1000 U/kg body weight of the subject. In some embodiments, the ADAMTS13 composition is administered to the subject at a dose of from 1000 U/kg to 4000 U/kg body weight of the subject. In some embodiments, the ADAMTS13 composition is administered to the subject at a dose of from 2000 U/kg to 4000 U/kg body weight of the subject.

In some embodiments, the ADAMTS13 composition is administered to the subject at a dose of about 10, 20, 30, 40, 50, 60, 70, 80, 90, 100, 125, 150, 200, 250, 300, 400, 500, 600, 700, 800, 900, 1000, 1500, 2000, 3000, or 4000 mg A13/kg of the subject. In some embodiments, the ADAMTS13 composition is administered to the subject at a dose of from 10 mg/kg to 2000 mg/kg body weight of the subject. In some embodiments, the ADAMTS13 composition is administered to the subject at a dose of from 10 mg/kg to 500 mg/kg body weight of the subject. In some embodiments, the ADAMTS13 composition is administered to the subject at a dose of from 20 mg/kg to 250 mg/kg body weight of the subject. In some embodiments, the ADAMTS13 composition is administered to the subject at a dose of from 50 mg/kg to 125 mg/kg body weight of the subject. In some embodiments, the ADAMTS13 composition is administered to the subject at a dose of from 10 mg/kg to 250 mg/kg body weight of the subject. In some embodiments, the ADAMTS13 composition is administered to the subject at a dose of from 10 mg/kg to 125 mg/kg body weight of the subject. In some embodiments, the ADAMTS13 composition is administered to the subject at a dose of from 10 mg/kg to 50 mg/kg body weight of the subject. In some embodiments, the ADAMTS13 composition is administered to the subject at a dose of from 20 mg/kg to 125 mg/kg body weight of the subject. In some embodiments, the ADAMTS13 composition is administered to the subject at a dose of from 20 mg/kg to 50 mg/kg body weight of the subject. In some embodiments, the ADAMTS13 composition is administered to the subject at a dose of from 50 mg/kg to 500 mg/kg body weight of the subject. In some embodiments, the ADAMTS13 composition is administered to the subject at a dose of from 50 mg/kg to 250 mg/kg body weight of the subject. In some embodiments, the ADAMTS13 composition is administered to the subject at a dose of from 50 mg/kg to 125 mg/kg body weight of the subject. In some embodiments, the ADAMTS13 composition is administered to the subject at a dose of from 125 mg/kg to 1000 mg/kg body weight of the subject. In some embodiments, the ADAMTS13 composition is administered to the subject at a dose of from 125 mg/kg to 500 mg/kg body weight of the subject. In some embodiments, the ADAMTS13 composition is administered to the subject at a dose of from 125 mg/kg to 250 mg/kg body weight of the subject. In some embodiments, the ADAMTS13 composition is administered to the subject at a dose of from 250 mg/kg to 2000 mg/kg body weight of the subject. In some embodiments, the ADAMTS13 composition is administered to the subject at a dose of from 250 mg/kg to 1000 mg/kg body weight of the subject. In some embodiments, the ADAMTS13 composition is administered to the subject at a dose of from 250 mg/kg to 500 mg/kg body weight of the subject. In some embodiments, the ADAMTS13 composition is administered to the subject at a dose of from 500 mg/kg to 2000 mg/kg body weight of the subject. In some embodiments, the ADAMTS13 composition is administered to the subject at a dose of from 1000 mg/kg to 2000 mg/kg body weight of the subject. In some embodiments, the ADAMTS13 composition is administered to the subject at a dose of from 2000 mg/kg to 4000 mg/kg body weight of the subject.

In some embodiments, the method includes administering a single dose of ADAMTS13 (or an ADAMTS13 agonist) to the subject. In some embodiment, the method includes administering a single dose of ADAMTS13 to the subject in response to a flare-up of an IBD (e.g., a particular subtype of IBD). In some embodiments, the method includes administration of multiple doses of ADAMTS13 to the subject. In some embodiments, the method includes repeated dosing of ADAMTS13 in response to a flare-up of an IBD (e.g., a particular subtype of IBD). In some embodiments, the repeated administration continues until alleviation, or partial alleviation, of a symptom of the IBD flare-up (e.g., until reduction or elimination of abdominal pain, rectal bleeding or blood in the subject's stool, loose bowel movements, loss of appetite, or abdominal inflammation). In some embodiments, the repeated dosing includes weekly dosing, twice weekly dosing, bi-daily (e.g., every other day) dosing, daily dosing, twice daily dosing, three times daily dosing, etc.

In some embodiments, the method includes repeated dosing of ADAMTS13 for managing an IBD (e.g., a particular subtype of IBD) or for managing a symptom of IBD (e.g., abdominal pain, rectal bleeding or blood in the subject's stool, loose bowel movements, loss of appetite, or abdominal inflammation). In some embodiments, the method includes repeated dosing of ADAMTS13 for prophylactic treatment of an IBD (e.g., a particular subtype of IBD) or for prophylactically treating a symptom of IBD (e.g., abdominal pain, rectal bleeding or blood in the subject's stool, loose bowel movements, loss of appetite, or abdominal inflammation). In some embodiments, the prophylactic ADAMTS13 treatment is for a subject having an elevated risk of developing IBD. In some embodiments, the repeated dosing includes bi-monthly (e.g., every other month) dosing, monthly dosing, twice monthly dosing, weekly dosing, twice weekly dosing, bi-daily (e.g., every other day) dosing, daily dosing, twice daily dosing, three times daily dosing, etc.

In certain embodiments, an effective dose of a composition comprising an agonist of ADAMTS13 as described herein can be administered to a patient once. In certain embodiments, an effective dose of a composition comprising an agonist of ADAMTS13 can be administered to a patient repeatedly. For systemic administration, subjects can be administered a therapeutic amount of a composition comprising an agonist of ADAMTS13, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. For systemic administration, subjects can be administered a therapeutic amount of a composition comprising an ADAMTS13 polypeptide (e.g. a recombinant ADAMTS13 polypeptide), such as, e.g. about 1000-5000 U/kg/day, about 2000-4500 U/kg/day, about 3000-4000 U/kg/day, or about 3500 U/kg/day. For systemic administration, subjects can be administered a therapeutic amount of a composition comprising an ADAMTS13 polypeptide (e.g. a recombinant ADAMTS13 polypeptide), such as, e.g. 1000-5000 U/kg/day, 2000-4500 U/kg/day, 3000-4000 U/kg/day, or 3500 U/kg/day.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the agonist of ADAMTS13. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising an agonist of ADAMTS13 can be administered over a period of time, such as over a 5 minute, 10 minute, 15 minute, 20 minute, or 25 minute period.

The dosage ranges for the administration of, e.g., an agonist of ADAMTS13, according to the methods described herein depend upon, for example, the form of an agonist of ADAMTS13, its potency, and the extent to which symptoms, markers, or indicators of a condition described herein are desired to be reduced, for example the percentage increase of ADAMTS13 levels, or the extent to which, for example, symptoms of colitis are desired to be reduced. The dosage should not be so large as to cause adverse side effects. Generally, the dosage will vary with the age, condition, and sex of the patient and can be determined by one of skill in the art. The dosage can also be adjusted by the individual physician in the event of any complication.

In some embodiments, an ADAMTS13 pharmaceutical composition includes one or more pharmaceutically acceptable carrier and/or diluent. In some embodiments, an ADAMTS13 pharmaceutical composition includes one or more additional active ingredients such as agents that stimulate ADAMTS13 production or secretion by the treated patient/subject, agents that inhibit the degradation of ADAMTS13 and thus prolong its half life (or alternatively glycosylated variants of ADAMTS13), agents that enhance ADAMTS13 activity (for example by binding to ADAMTS13, thereby inducing an activating conformational change), or agents that inhibit ADAMTS13 clearance from circulation, thereby increasing its plasma concentration.

In vitro and animal model assays are provided herein which allow the assessment of a given dose of an agonist of ADAMTS13. By way of non-limiting example, the effects of a dose of an agonist of ADAMTS13 can be assessed by administering the agonist to ADAMTS13−/− mice, e.g. with or without secondary colitis-inducing factors. The agonist can be administered, e.g. by intravenous injection. Efficacy can be measured by, e.g., inflammation, IL-6 levels, percent weight loss, colitis scores, and VWF levels.

Treatment Upon Diagnosis

In some embodiments, the methods described herein include: (a) diagnosing IBD in a subject, and (b) administering ADAMTS13 (or an ADAMTS13 agonist) to the subject in response to a positive IBD diagnosis. In some embodiments, the method includes: (a) diagnosing IBD in a subject, and (b) assigning treatment including administration of ADAMTS13 (or an ADAMTS13 agonist) to the subject in response to a positive IBD diagnosis.

In some embodiments, the methods described herein include: (a) identifying a clinical criterion associated with IBD in a subject, and (b) administering ADAMTS13 (or an ADAMTS13 agonist) to the subject in response to identifying the clinical criterion. In some embodiments, the method includes: (a) identifying a clinical criterion associated with IBD in a subject, and (b) assigning treatment including administration of ADAMTS13 (or an ADAMTS13 agonist) to the subject in response to identifying the clinical criterion.

In some embodiments, the methods described herein include: (a) diagnosing an IBD flare-up in a subject, and (b) administering ADAMTS13 (or an ADAMTS13 agonist) to the subject in response to diagnosing the IBD flare-up. In some embodiments, the method includes: (a) diagnosing an IBD flare-up in a subject, and (b) assigning treatment including administration of ADAMTS13 (or an ADAMTS13 agonist) to the subject in response to diagnosing the IBD flare-up.

Co-Administration

The methods described herein can further comprise administering a second agent and/or treatment to the subject, e.g. as part of a combinatorial therapy. Non-limiting examples can include anti-inflammatory (e.g. aminosalicylates or steroids), immunosuppressant agents, cyclosporine, and corticosteroids.

In some embodiments, the methods provided herein include co-administration of ADAMTS13 with a conventional treatment for an inflammatory bowel disease, for example, an anti-inflammatory agent (e.g., a steroid, such as a corticosteroid steroid) or immunosuppressant. In some embodiments, the method includes co-administration of ADAMTS13 with a conventional treatment for a flare-up of an inflammatory bowel disease, for example, an anti-inflammatory agent (e.g., a steroid, such as a corticosteroid steroid) or immunosuppressant.

In some embodiments, the methods provided herein include coadministration of ADAMTS13 with a conventional treatment for ulcerative colitis, for example, an aminosalicylate, a corticosteroid, azathioprine, mercapopurine, cyclosporine, a TNF inhibitor (e.g., an anti-TNF-alpha monoclonal antibody, such as infliximab, adalimumab, or golimumab), or an integrin α₄β₇ inhibitor (e.g., an anti-integrin α₄β₇ monoclonal antibody, such as vedolimumab).

In some embodiments, the methods provided herein include coadministration of ADAMTS13 with a conventional treatment for Crohn's disease, for example, an aminosalicylate (e.g., sulfasalazine or mesalamine), a corticosteroid, azathioprine, mercapopurine, cyclosporine, a TNF inhibitor (e.g., an anti-TNF-alpha monoclonal antibody, such as infliximab, adalimumab, or certolizumab), an integrin α₄β₇ inhibitor (e.g., an anti-integrin α₄β₇ monoclonal antibody, such as vedolimumab), an integrin α₅β₃ inhibitor (e.g., an anti-integrin α₄β₇ monoclonal antibody, such as natalizumab), methotrexate, tacrolimus, an interleukin 12/interleukin 23 inhibitor (e.g., an anti-interleukin 12/interleukin 23 monoclonal antibody, such as ustekinumab), or an antibody (e.g., metronidazole or ciprofloxacin).

### Specific Embodiments

In some embodiments, a method is provided for treating Crohn's disease by administering an ADAMTS13 polypeptide to a subject. In some embodiments, the method includes administering ADAMTS13 to a subject experiencing a flare-up of Crohn's disease. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of Crohn's disease or a flare-up thereof. In some embodiments, the method includes co-administration of ADAMTS13 and an immunosuppressant for treatment of Crohn's disease, or a flare-up thereof. In some embodiments, the method includes intravenous administration of ADAMTS13 for treatment of Crohn's disease, or a flare-up thereof. In some embodiments, the method includes subcutaneous administration of ADAMTS13 for treatment of Crohn's disease, or a flare-up thereof.

In some embodiments, a method is provided for treating ulcerative colitis by administering an ADAMTS13 polypeptide to a subject. In some embodiments, the method includes administering ADAMTS13 to a subject experiencing a flare-up of ulcerative colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of ulcerative colitis or a flare-up thereof. In some embodiments, the method includes co-administration of ADAMTS13 and an immunosuppressant for treatment of ulcerative colitis, or a flare-up thereof. In some embodiments, the method includes intravenous administration of ADAMTS13 for treatment of ulcerative colitis, or a flare-up thereof. In some embodiments, the method includes subcutaneous administration of ADAMTS13 for treatment of ulcerative colitis, or a flare-up thereof.

In some embodiments, a method is provided for treating an idiopathic colitis by administering an ADAMTS13 polypeptide to a subject. In some embodiments, the method includes administering ADAMTS13 to a subject experiencing a flare-up of an idiopathic colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of an idiopathic colitis or a flare-up thereof. In some embodiments, the method includes co-administration of ADAMTS13 and an immunosuppressant for treatment of an idiopathic colitis, or a flare-up thereof. In some embodiments, the method includes intravenous administration of ADAMTS13 for treatment of an idiopathic colitis, or a flare-up thereof. In some embodiments, the method includes subcutaneous administration of ADAMTS13 for treatment of an idiopathic colitis, or a flare-up thereof.

In some embodiments, a method is provided for treating microscopic colitis by administering an ADAMTS13 polypeptide to a subject. In some embodiments, the method includes administering ADAMTS13 to a subject experiencing a flare-up of microscopic colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of microscopic colitis or a flare-up thereof. In some embodiments, the method includes co-administration of ADAMTS13 and an immunosuppressant for treatment of microscopic colitis, or a flare-up thereof. In some embodiments, the method includes intravenous administration of ADAMTS13 for treatment of microscopic colitis, or a flare-up thereof. In some embodiments, the method includes subcutaneous administration of ADAMTS13 for treatment of microscopic colitis, or a flare-up thereof.

In some embodiments, a method is provided for treating lymphocytic colitis by administering an ADAMTS13 polypeptide to a subject. In some embodiments, the method includes administering ADAMTS13 to a subject experiencing a flare-up of lymphocytic colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of lymphocytic colitis or a flare-up thereof. In some embodiments, the method includes co-administration of ADAMTS13 and an immunosuppressant for treatment of lymphocytic colitis, or a flare-up thereof. In some embodiments, the method includes intravenous administration of ADAMTS13 for treatment of lymphocytic colitis, or a flare-up thereof. In some embodiments, the method includes subcutaneous administration of ADAMTS13 for treatment of lymphocytic colitis, or a flare-up thereof.

In some embodiments, a method is provided for treating collagenous colitis by administering an ADAMTS13 polypeptide to a subject. In some embodiments, the method includes administering ADAMTS13 to a subject experiencing a flare-up of collagenous colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of collagenous colitis or a flare-up thereof. In some embodiments, the method includes co-administration of ADAMTS13 and an immunosuppressant for treatment of collagenous colitis, or a flare-up thereof. In some embodiments, the method includes intravenous administration of ADAMTS13 for treatment of collagenous colitis, or a flare-up thereof. In some embodiments, the method includes subcutaneous administration of ADAMTS13 for treatment of collagenous colitis, or a flare-up thereof.

In some embodiments, a method is provided for treating an iatrogenic colitis by administering an ADAMTS13 polypeptide to a subject. In some embodiments, the method includes administering ADAMTS13 to a subject experiencing a flare-up of an iatrogenic colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of an iatrogenic colitis or a flare-up thereof. In some embodiments, the method includes co-administration of ADAMTS13 and an immunosuppressant for treatment of an iatrogenic colitis, or a flare-up thereof. In some embodiments, the method includes intravenous administration of ADAMTS13 for treatment of an iatrogenic colitis, or a flare-up thereof. In some embodiments, the method includes subcutaneous administration of ADAMTS13 for treatment of an iatrogenic colitis, or a flare-up thereof.

In some embodiments, a method is provided for treating diversion colitis by administering an ADAMTS13 polypeptide to a subject. In some embodiments, the method includes administering ADAMTS13 to a subject experiencing a flare-up of diversion colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of diversion colitis or a flare-up thereof. In some embodiments, the method includes co-administration of ADAMTS13 and an immunosuppressant for treatment of diversion colitis, or a flare-up thereof. In some embodiments, the method includes intravenous administration of ADAMTS13 for treatment of diversion colitis, or a flare-up thereof. In some embodiments, the method includes subcutaneous administration of ADAMTS13 for treatment of diversion colitis, or a flare-up thereof.

In some embodiments, a method is provided for treating neutropenic enterocolitis by administering an ADAMTS13 polypeptide to a subject. In some embodiments, the method includes administering ADAMTS13 to a subject experiencing a flare-up of neutropenic enterocolitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of neutropenic enterocolitis or a flare-up thereof. In some embodiments, the method includes co-administration of ADAMTS13 and an immunosuppressant for treatment of neutropenic enterocolitis, or a flare-up thereof. In some embodiments, the method includes intravenous administration of ADAMTS13 for treatment of neutropenic enterocolitis, or a flare-up thereof. In some embodiments, the method includes subcutaneous administration of ADAMTS13 for treatment of neutropenic enterocolitis, or a flare-up thereof.

In some embodiments, a method is provided for treating disinfectant colitis by administering an ADAMTS13 polypeptide to a subject. In some embodiments, the method includes administering ADAMTS13 to a subject experiencing a flare-up of disinfectant colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of disinfectant colitis or a flare-up thereof. In some embodiments, the method includes co-administration of ADAMTS13 and an immunosuppressant for treatment of disinfectant colitis, or a flare-up thereof. In some embodiments, the method includes intravenous administration of ADAMTS13 for treatment of disinfectant colitis, or a flare-up thereof. In some embodiments, the method includes subcutaneous administration of ADAMTS13 for treatment of disinfectant colitis, or a flare-up thereof.

In some embodiments, a method is provided for treating corrosive colitis by administering an ADAMTS13 polypeptide to a subject. In some embodiments, the method includes administering ADAMTS13 to a subject experiencing a flare-up of corrosive colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of corrosive colitis or a flare-up thereof. In some embodiments, the method includes co-administration of ADAMTS13 and an immunosuppressant for treatment of corrosive colitis, or a flare-up thereof. In some embodiments, the method includes intravenous administration of ADAMTS13 for treatment of corrosive colitis, or a flare-up thereof. In some embodiments, the method includes subcutaneous administration of ADAMTS13 for treatment of corrosive colitis, or a flare-up thereof.

In some embodiments, a method is provided for treating nonsteroidal anti-inflammatory drug or salicylate-induced colitis by administering an ADAMTS13 polypeptide to a subject. In some embodiments, the method includes administering ADAMTS13 to a subject experiencing a flare-up of nonsteroidal anti-inflammatory drug or salicylate-induced colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of nonsteroidal anti-inflammatory drug or salicylate-induced colitis or a flare-up thereof. In some embodiments, the method includes co-administration of ADAMTS13 and an immunosuppressant for treatment of nonsteroidal anti-inflammatory drug or salicylate-induced colitis, or a flare-up thereof. In some embodiments, the method includes intravenous administration of ADAMTS13 for treatment of nonsteroidal anti-inflammatory drug or salicylate-induced colitis, or a flare-up thereof. In some embodiments, the method includes subcutaneous administration of ADAMTS13 for treatment of nonsteroidal anti-inflammatory drug or salicylate-induced colitis, or a flare-up thereof.

In some embodiments, a method is provided for treating toxic epidermal necrolysis by administering an ADAMTS13 polypeptide to a subject. In some embodiments, the method includes administering ADAMTS13 to a subject experiencing a flare-up of toxic epidermal necrolysis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of toxic epidermal necrolysis or a flare-up thereof. In some embodiments, the method includes co-administration of ADAMTS13 and an immunosuppressant for treatment of toxic epidermal necrolysis, or a flare-up thereof. In some embodiments, the method includes intravenous administration of ADAMTS13 for treatment of toxic epidermal necrolysis, or a flare-up thereof. In some embodiments, the method includes subcutaneous administration of ADAMTS13 for treatment of toxic epidermal necrolysis, or a flare-up thereof.

In some embodiments, a method is provided for treating a chemical-induced colitis by administering an ADAMTS13 polypeptide to a subject. In some embodiments, the method includes administering ADAMTS13 to a subject experiencing a flare-up of a chemical-induced colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of a chemical-induced colitis or a flare-up thereof. In some embodiments, the method includes co-administration of ADAMTS13 and an immunosuppressant for treatment of a chemical-induced colitis, or a flare-up thereof. In some embodiments, the method includes intravenous administration of ADAMTS13 for treatment of a chemical-induced colitis, or a flare-up thereof. In some embodiments, the method includes subcutaneous administration of ADAMTS13 for treatment of a chemical-induced colitis, or a flare-up thereof.

In some embodiments, a method is provided for treating ischemic colitis by administering an ADAMTS13 polypeptide to a subject. In some embodiments, the method includes administering ADAMTS13 to a subject experiencing a flare-up of ischemic colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of ischemic colitis or a flare-up thereof. In some embodiments, the method includes co-administration of ADAMTS13 and an immunosuppressant for treatment of ischemic colitis, or a flare-up thereof. In some embodiments, the method includes intravenous administration of ADAMTS13 for treatment of ischemic colitis, or a flare-up thereof. In some embodiments, the method includes subcutaneous administration of ADAMTS13 for treatment of ischemic colitis, or a flare-up thereof.

In some embodiments, a method is provided for treating an infectious colitis by administering an ADAMTS13 polypeptide to a subject. In some embodiments, the method includes administering ADAMTS13 to a subject experiencing a flare-up of an infectious colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of an infectious colitis or a flare-up thereof. In some embodiments, the method includes co-administration of ADAMTS13 and an immunosuppressant for treatment of an infectious colitis, or a flare-up thereof. In some embodiments, the method includes intravenous administration of ADAMTS13 for treatment of an infectious colitis, or a flare-up thereof. In some embodiments, the method includes subcutaneous administration of ADAMTS13 for treatment of an infectious colitis, or a flare-up thereof.

In some embodiments, a method is provided for treating Clostridium difficile colitis by administering an ADAMTS13 polypeptide to a subject. In some embodiments, the method includes administering ADAMTS13 to a subject experiencing a flare-up of Clostridium difficile colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of Clostridium difficile colitis or a flare-up thereof. In some embodiments, the method includes co-administration of ADAMTS13 and an immunosuppressant for treatment of Clostridium difficile colitis, or a flare-up thereof. In some embodiments, the method includes intravenous administration of ADAMTS13 for treatment of Clostridium difficile colitis, or a flare-up thereof. In some embodiments, the method includes subcutaneous administration of ADAMTS13 for treatment of Clostridium difficile colitis, or a flare-up thereof.

In some embodiments, a method is provided for treating eosinophilic colitis by administering an ADAMTS13 polypeptide to a subject. In some embodiments, the method includes administering ADAMTS13 to a subject experiencing a flare-up of eosinophilic colitis. In some embodiments, the method includes intravenous administration of ADAMTS13 to the subject. In other embodiments, the method includes subcutaneous administration of ADAMTS13 to the subject. In some embodiments, the method comprises administering from 20 U A13/kg to 1000 U A13/kg of the subject. In some embodiments, the method comprises administering from 40 U A13/kg to 500 U A13/gk of the subject. In some embodiments, the method includes co-administration of ADAMTS13 and an anti-inflammatory agent (e.g., a steroid, such as a corticosteroid) for treatment of eosinophilic colitis or a flare-up thereof. In some embodiments, the method includes co-administration of ADAMTS13 and an immunosuppressant for treatment of eosinophilic colitis, or a flare-up thereof. In some embodiments, the method includes intravenous administration of ADAMTS13 for treatment of eosinophilic colitis, or a flare-up thereof. In some embodiments, the method includes subcutaneous administration of ADAMTS13 for treatment of eosinophilic colitis, or a flare-up thereof.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating an inflammatory bowel disease (IBD), the method comprising administering ADAMTS13 to a subject in need thereof.
2. A method for treating a flare-up of an inflammatory bowel disease (IBD), the method comprising administering ADAMTS13 to a subject in need thereof
3. A method of treating a symptom of an inflammatory bowel disease (IBD), the method comprising administering ADAMTS13 to a subject in need thereof.
4. The method of paragraph 3, wherein the symptom of inflammatory bowel disease (IBD) is inflammation of the colon or small intestine.
5. A method of preventing a thrombosis, ischemia, and/or pulmonary embolism associated with IBD, the method comprising administering ADAMTS13 to a subject in need thereof
6. The method of any of paragraphs 1-5, wherein the ADAMTS13 is administered intravenously.
7. The method of any of paragraphs 1-5, wherein the ADAMTS13 is administered subcutaneously.
8. The method of any of paragraphs 1-7, wherein the ADAMTS13 is a human ADAMTS13 polypeptide.
9. The method of any of 1-8, wherein the ADAMTS13 is recombinant ADAMTS13.
10. The method of any of paragraphs 1-8, wherein the ADAMTS13 is plasma-derived ADAMTS13.
11. The method of any of paragraphs 1-10, wherein the ADAMTS13 is administered at a dosage of from about 20 U/kg to about 1000 U/kg.
12. The method of any of paragraphs 1-10, wherein the ADAMTS13 is administered at a dosage of from about 40 U/kg to about 500 U/kg.
13. The method of any of paragraphs 1-10, wherein the ADAMTS13 is administered at a dosage of from about 40 U/kg to about 250 U/kg.
14. The method of any of paragraphs 1-13, wherein the ADAMTS13 is administered at least every 2 days.
15. The method of any of paragraphs 1-13, wherein the ADAMTS13 is administered daily.
16. The method of any of paragraphs 1-15, comprising:
   (a) determining a level of ADAMTS13 expression in the subject; and
   (b) administering ADAMTS13 to the subject if the level of ADAMTS13 expression is reduced relative to the average level of ADAMTS13 expression in subjects without inflammatory bowel disease.
17. The method of any of paragraphs 1-15, comprising:
   (a) determining a level of ADAMTS13 activity in the subject; and
   (b) administering ADAMTS13 to the subject if the level of ADAMTS13 activity is reduced relative to the average level of ADAMTS13 activity in subjects without inflammatory bowel disease.
18. The method of any of paragraphs 1 and 3-17, wherein the subject is administered ADAMTS13 in response to a flare-up of the IBD.
19. The method of any of paragraphs 1-18, wherein the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.
20. The method of paragraph 19, wherein the idiopathic colitis is microscopic colitis, lymphocytic colitis, or collagenous colitis.
21. The method of paragraph 19, wherein the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis
22. The method of paragraph 19, wherein the infectious colitis is Clostridium difficile colitis.
23. A composition for use in the treatment of an inflammatory bowel disease (IBD), the composition comprising ADAMTS13.
24. A composition for use in treatment of a flare-up of an inflammatory bowel disease (IBD), the composition comprising ADAMTS13.
25. A composition for use in the treatment of a symptom of an inflammatory bowel disease (IBD), the composition comprising ADAMTS13.
26. The composition of paragraph 25, wherein the symptom of inflammatory bowel disease (IBD) is inflammation of the colon or small intestine.
27. A composition for use in the prevention of a thrombosis, ischemia, and/or pulmonary embolism associated with IBD, the composition comprising ADAMTS13.
28. The composition of any of paragraphs 23-27, wherein the composition is formulated for intravenous administration.
29. The composition of any of paragraphs 23-27, wherein the composition is formulated for subcutaneous administration.
30. The composition of any of paragraphs 23-28, wherein the ADAMTS13 is a human ADAMTS13 polypeptide.
31. The composition of any of paragraphs 23-29, wherein the ADAMTS13 is recombinant ADAMTS13.
32. The composition of any of paragraphs 23-29, wherein the ADAMTS13 is plasma-derived ADAMTS13.
33. The composition of any of paragraphs 23-31, wherein the ADAMTS13 is formulated at a dosage of from about 20 U/kg to about 1000 U/kg.
34. The composition of any of paragraphs 23-31, wherein the ADAMTS13 is formulated at a dosage of from about 40 U/kg to about 500 U/kg.
35. The composition of any of paragraphs 23-31, wherein the ADAMTS13 is formulated at a dosage of from about 40 U/kg to about 250 U/kg.
36. The composition of any of paragraphs 23-35, wherein the ADAMTS13 is formulated for administration at least every 2 days.
37. The composition of any of paragraphs 23-35, wherein the ADAMTS13 is formulated for daily administration.
38. The composition of any of paragraphs 23 and 25-37, wherein the ADAMTS13 is formulated for administration in response to a flare-up of IBD.
39. The composition of any of paragraphs 23-38, wherein the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.
40. The composition of paragraph 39, wherein the idiopathic colitis is microscopic colitis, lymphocytic colitis, or collagenous colitis.
41. The composition of paragraph 39, wherein the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis
42. The composition of paragraph 39, wherein the infectious colitis is Clostridium difficile colitis.
43. Use of ADAMTS13 for the manufacture of a medicament for the treatment of inflammatory bowel disease (IBD).
44. Use of ADAMTS13 for the manufacture of a medicament for the treatment of a flare-up of inflammatory bowel disease (IBD).
45. Use of ADAMTS13 for the manufacture of a medicament for the treatment of a symptom of an inflammatory bowel disease.
46. The Use of paragraph 45, wherein the symptom of inflammatory bowel disease (IBD) is inflammation of the colon or small intestine.
47. Use of ADAMTS13 for the manufacture of a medicament for the prevention of a thrombosis, ischemia, and/or pulmonary embolism associated with IBD, the composition comprising ADAMTS13.
48. The use of any of paragraphs 43-47, wherein the medicament is formulated for intravenous administration.
49. The use of any of paragraphs 43-47, wherein the medicament is formulated for subcutaneous administration.
50. The use of any of paragraphs 43-49, wherein the ADAMTS13 is a human ADAMTS13 polypeptide.
51. The use of any of paragraphs 43-50, wherein the ADAMTS13 is recombinant ADAMTS13.
52. The use of any of paragraphs 43-50, wherein the ADAMTS13 is plasma-derived ADAMTS13.
53. The use of any of paragraphs 43-52, wherein the ADAMTS13 is formulated at a dosage of from about 20 U/kg to about 1000 U/kg.
54. The use of any of paragraphs 43-52, wherein the ADAMTS13 is formulated at a dosage of from about 40 U/kg to about 500 U/kg.
55. The use of any of paragraphs 43-52, wherein the ADAMTS13 is formulated at a dosage of from about 40 U/kg to about 250 U/kg.
56. The use of any of paragraphs 43-55, wherein the ADAMTS13 is formulated for administration at least every 2 days.
57. The use of any of paragraphs 43-55, wherein the ADAMTS13 is formulated for daily administration.
58. The use of any of paragraphs 43 and 45-57, wherein the ADAMTS13 is formulated for administration in response to a flare-up of IBD.
59. The use of any of paragraphs 43-58, wherein the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.
60. The use of paragraph 59, wherein the idiopathic colitis is microscopic colitis, lymphocytic colitis, or collagenous colitis.
61. The use of paragraph 59, wherein the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis
62. The use of paragraph 59, wherein the infectious colitis is Clostridium difficile colitis.

### Examples

### Example 1: ADAMTS13 Deficiency Worsens Murine Colitis and Exogenous ADAMTS13 Administration can Decrease Colitis Severity in Wild Type Mice

As described herein, ADAMTS13-deficient mice have more severe colitis demonstrated by increased weight loss, anemia, clinical and histologic colitis severity. Administration of ADAMTS13 to wild type mice with colitis decreases colitis severity, and IL-6 without worsening anemia.

Inflammatory Bowel Disease (IBD) affects 1.2 million people in the US with a rising incidence. Patients with IBD have an elevated risk of thrombosis that correlates with worsening of disease. The pathogenesis of IBD and its increased thrombotic risk is incompletely understood. Ultra large von Willebrand Factor (ULVWF) multimers are released from activated endothelium in response to inflammatory stimuli and recruits both platelets and leukocytes. ADAMTS13 is an enzyme that cleaves the highly adhesive VWF into less bioactive smaller multimers releasing them into circulation. Mice deficient in ADAMTS13 (ADAMTS13−/−) have increased leukocyte rolling and adhesion at baseline which increases with inflammatory stimuli. Since intestinal inflammation is a hallmark of IBD, we hypothesized that ADAMTS13−/− mice would have more severe colitis compared to wild-type (WT). Chemical (Dextran Sodium Sulfate) colitis was induced in ADAMTS13−/− and WT mice. Colitis was worse in ADAMTS13−/− mice demonstrated by increased weight loss, worse anemia clinical and histologic colitis severity. ADAMTS13−/− mice had increased VWF release at inflamed colonic sites and the majority of mice showed one or more submucosal colonic thrombi. Exogenous ADAMTS13 administration in WT mice decreased colitis severity (reduced weight loss and clinical colitis scores), without worsening colonic bleeding, and resulted in less plasma IL-6, a pro-inflammatory cytokine important in IBD. This study demonstrates that ADAMTS13 deficiency worsens colitis most likely through increased platelet and leukocyte recruitment and microthrombi formation. Furthermore, ADAMTS13 administration is of therapeutic value by both decreasing colonic inflammation and preventing thrombosis in IBD, thus improving outcome.

Furthermore, ADAMTS13 levels and activity are inversely correlated with disease activity and acute phase reactants such as C-reactive protein and fibrinogen (23). Therefore ADAMTS13 deficiency or acquired antibody mediated ADAMTS13 inhibition could contribute to the increased incidence of thrombosis in this population.

Described herein is the evaluation of the role of ADAMTS13 in Dextran Sodium Sulfate (DSS)-induced colitis by comparing ADAMTS13−/− and wild type (WT) mice. The results described herein demonstrate that ADAMTS13 deficiency leads to the accumulation of VWF-rich thrombi in colonic submucosal vessels, subsequent increasingly inflamed colonic tissue, and a worse colitis phenotype. Moreover, it is demonstrated that treatment of WT mice with rhADAMTS13 ameliorates colitis which identifies ADAMTS13 as a drug for treating colitis.

Materials and Methods:

Animals:

ADAMTS13−/− {Motto, 2005 #924} and wild-type (WT) mice were on a C57BL/6J background. Animals were all 6-8 weeks old males, weight matched, who were in house bred wild type mice, and the majority from het/het crossess the remaining from in house bred WT C57BL/6J. All experimental procedures were reviewed and approved by the Institutional Animal Care and Use Committee of Boston Children's Hospital.

DSS Colitis Model:

Acute DSS colitis was induced by administration of DSS salt (3% wt/vol, mol. wt: 36-50 kDa; MP Biomedicals) in autoclaved drinking water for 10 days as previously described (25). Fresh DSS was administered on day 3 and 6. Daily weight and clinical colitis scores were measured using an established scoring system based on percentage weight loss (<10%=1, >10%=2, >20%=3), onset of diarrhea(1), bloody diarrhea(2), hunched posture (1) and rectal prolapse (1) for a maximum score of 7 (25).

Recombinant Human ADAMTS13 (rhADAMTS13) Treatment.

Treatment with rhADAMTS13 (or saline vehicle) by retro-orbital intravenous injections on day 3-10 at a dose of 3460 U/kg every 24 hours a concentration shown to decrease leukocyte recruitment in myocardial ischemia reperfusion injury (26).

Analysis of Peripheral Blood:

Blood was collected via the retro-orbital sinus into EDTA-coated capillary tubes and was analyzed by a Hemavet 950FS (Drew Scientific) for complete blood counts.

Plasma VWF, IL-6, IL-10, Soluble P-Selectin (sPsel) and Thrombin Anti-Thrombin Complexes (TAT):

Plasma VWF levels were quantified by ELISA on day 10 (27). VWF levels in plasma of WT or ADAMTS13−/− mice were calculated and shown as fold increase over pooled plasma VWF levels of untreated WT or untreated ADAMTS13−/− mice, respectively.

Tissue Preparation and Analysis:

Mice were anesthetized with isofluorane and terminally bled from the retro-orbital sinus. Entire spleens were harvested and weighed. Colon length was measured from anal verge to cecum then colons were cut longitudinally and Swiss rolled so the entire colon could be evaluated {Wirtz, 2007 #778}. Colons were fixed in zinc fixative (100 mM Tris-HCl containing 37 mM zinc chloride, 23 mM zinc acetate, and 3.2 mM calcium acetate). Paraffin-embedded sections were stained with H&E and scored for colitis severity using an established scoring system (28) by an individual blinded to the study group. Human colon tissue biopsies were in embedded in OCT, snap frozen, cryostat sectioned and fixed in zinc as described above.

Immunostaining and Fluorescence Microscopy:

Tissue sections were washed with PBS and permeabilized (0.1% Triton X-100, 0.1% sodium citrate) for 10 min at 4° C. Samples were blocked with 3% (wt/vol) BSA for 90 min at 37° C., rinsed, and then incubated overnight at 4° C. or for 1 h at 37° C. in primary antibody dilution buffer containing 0.3% BSA and rabbit antihuman VWF (DAKO, 1:500) and rat antimouse CD31 (1:250, Biolegend). After several washes, samples were incubated for 2 h at room temperature in antibody dilution buffer containing Alexa Fluor™-conjugated secondary antibodies in 0.3% BSA in PBS: goat anti-rat Ig (IgG) (Alexa555, 2 μg/mL, Biolegend) donkey anti-rabbit IgG (Alexa488, 1.5 μg/mL, Biolegend), or donkey anti-sheep IgG (Alexa568, 2 μg/mL; Invitrogen). DNA was counterstained with 1 μg/mL Hoechst 33342 and slides were coverslipped with Fluoromount™ gel (Electron Microscopy Sciences). Fluorescent images were acquired using an Axiovert 200™ wide field fluorescence microscope (Zeiss) in conjunction with an Axiocam™ MRm monochromatic CCD camera (Zeiss) and analyzed with Zeiss Axiovision™ software. All channels were acquired in grayscale and pseudo colored using Zeiss Axiovision™ or ImageJ™ software (National Institutes of Health). VWF in colon tissue area was quantified by assessing VWF area divided by DNA area which stained all nuclei so it estimated entire tissue area in 5 separate 20× images, initially selecting random areas and then only severely affected areas, to quantify VWF staining.

Results

ADAMTS13 Deficiency Results in a More Severe DSS Colitis Phenotype.

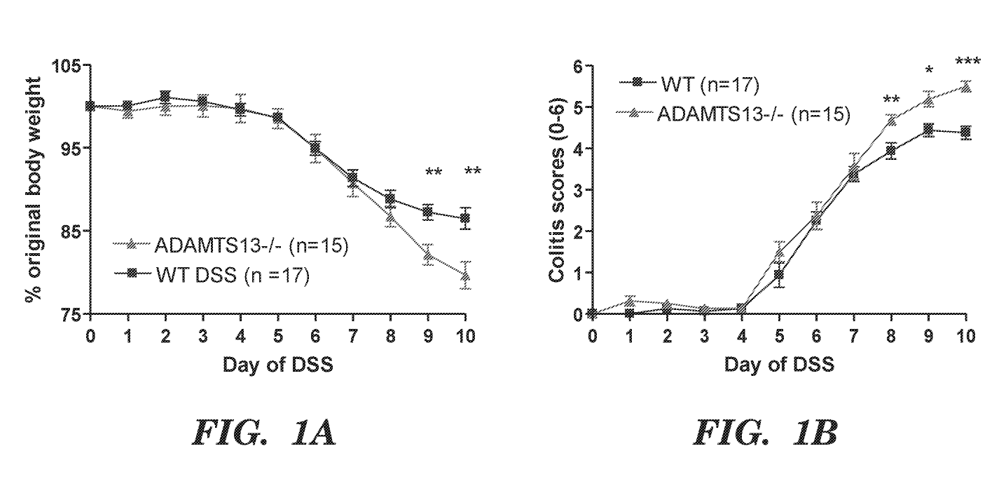

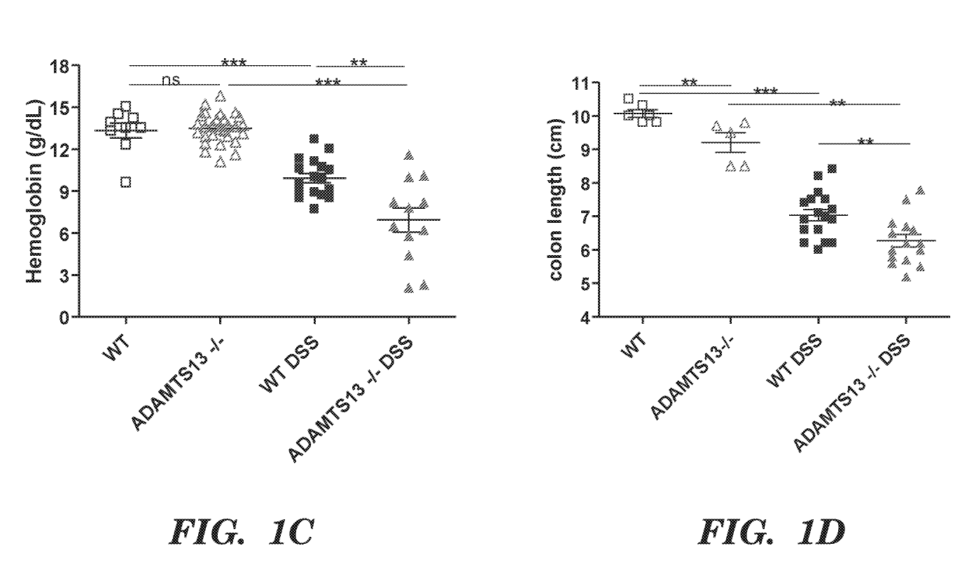

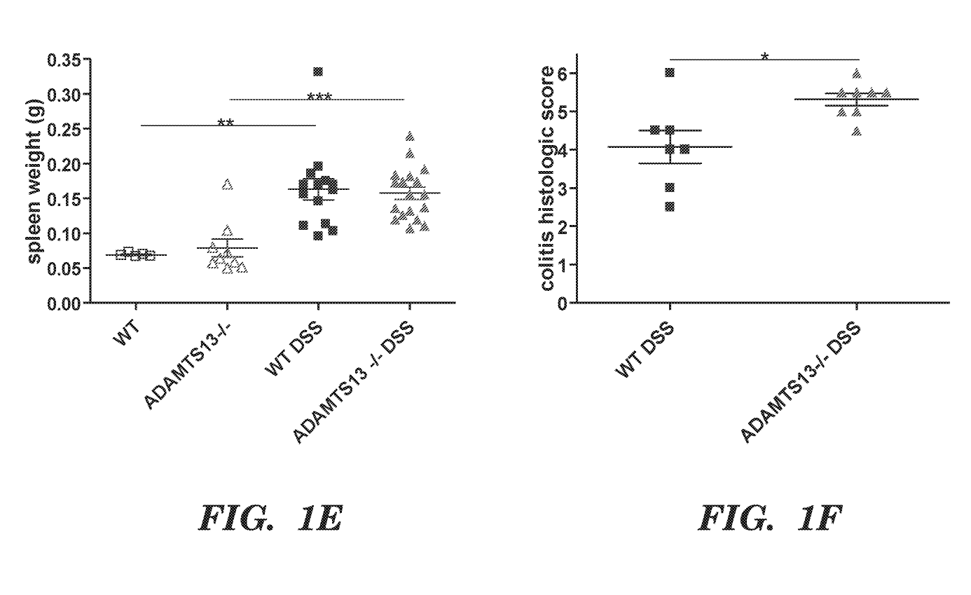

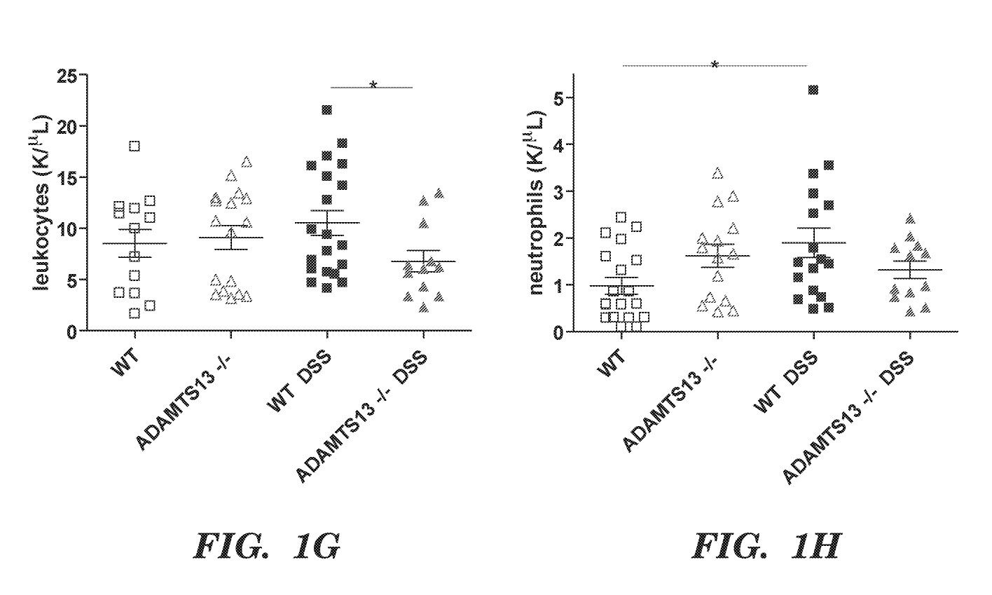

Initial body weight did not differ between ADAMTS13−/− and wild type mice nor did they have diarrhea at baseline. In three separate experiments ADAMTS13−/− mice (n=15) with DSS induced colitis lost more weight compared to WT (n=17) on days 9 (p<0.005) and 10 (p<0.0005) of colitis when it is most severe (FIG. 1A). Clinical colitis scores were also worse on days 8-10 (FIG. 1B; p<0.005, p<0.05, p<0.0005 respectively) this was attributed to both more weight loss, earlier onset of bloody diarrhea, and hunched body habitus. Hemoglobin concentration did not differ significantly at baseline prior to colitis induction suggesting ADAMTS13 deficiency did not result in clinically significant bleeding without mucosal injury (FIG. 1C). In colitis, hemoglobin concentration decreases due to blood loss in stool. ADAMTS13−/− mice developed worse anemia (p<0.005) compared to WT mice with colitis (FIG. 1C).

With colonic inflammation the colon becomes edematous and shortens (25). ADAMTS13−/− mice had slightly shorter colons compared to WT even without colitis (FIG. 1D, p<0.005). Because of this slight difference microscopic inflammation was looked for in H&E stained colon sections from untreated ADAMTS13−/− mice but no evidence of inflammation was found (data not shown). After induction of DSS colitis, colons from ADAMTS13−/− became significantly shorter compared to those from WT (p<0.005) suggesting more severe colitis in ADAMTS13−/− mice.

Spleen weight is a marker of systemic inflammation in colitis mouse models. Spleen weight increased significantly in both WT and ADAMTS13−/− with colitis (p<0.0005) however it did not differ between ADAMTS13−/− and WT (FIG. 1E). This and the above observations confirm the induction of severe colitis in both genotypes of mice.

Entire H+E stained Swiss rolled colons were scored for histologic colitis severity using an established scoring system (28) by an individual blinded to the study groups. ADAMTS13−/− colons had more histologic evidence of inflammation compared to WT with representative sections shown (data not shown). H+E stained colon section from WT and ADAMTS13−/− mice without colitis, demonstrated normal crypt architecture with neatly aligned crypts whose bases are adjacent to the muscularis mucosa, abundant clear mucous filled goblet cells, preserved epithelial barrier with epithelial cells and their basolateral nuclei, no mucosal, submucosal or muscularis mucosal edema. WT mouse colon with DSS demonstrated distortion of crypt architecture (branching of the normally test tube shaped crypts), loss of goblet cells previously seen within crypts, increased leukocytes infiltrating the lamina propria between the crypts. There are also more leukocytes and edema in the submucosa, which widens the submucosal space. An intact epithelial barrier remains. With increased disease severity, there is complete loss of crypt architecture, increased leukocyte infiltration the epithelial barrier is maintained in WT. ADAMTS13−/− colon sections show similar features as wild type but are more severe with increased crypt distortion in the middle panel and then complete loss of crypt architecture, increased leukocyte infiltration, a more widely expanded submucosal space, and transmural inflammation and ulceration with a ragged disorganized epithelial barrier.

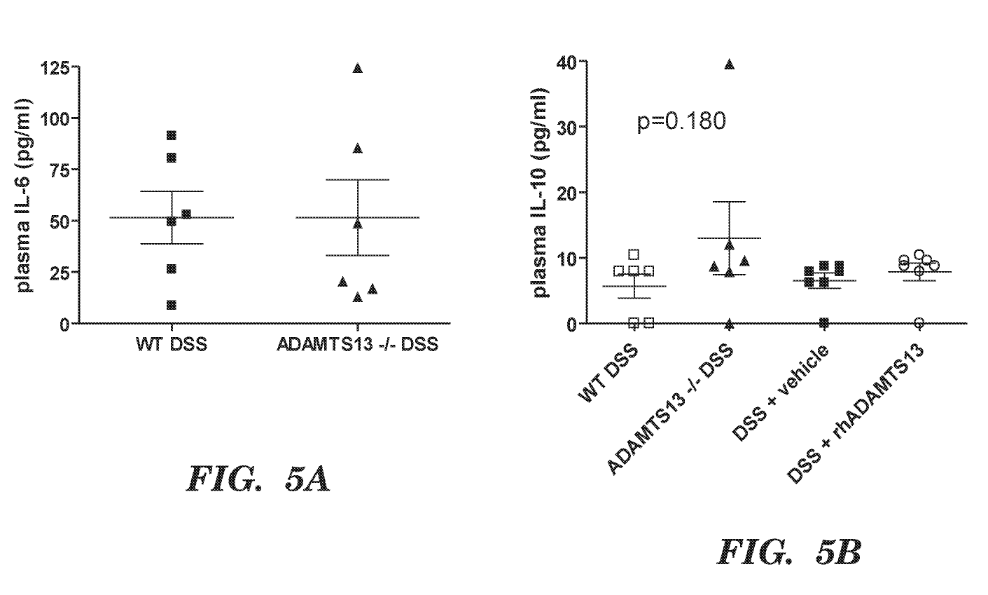

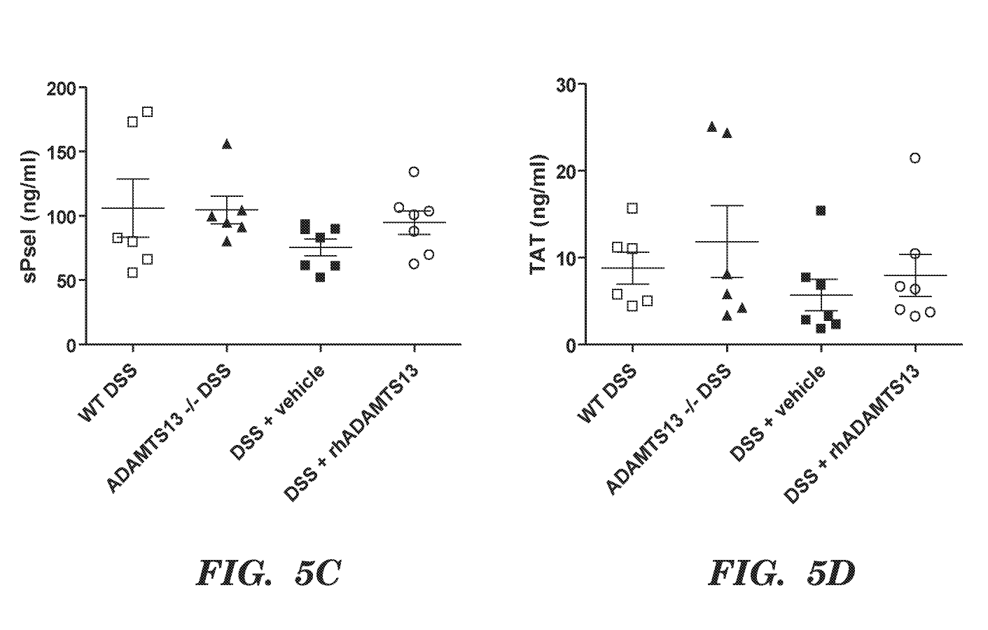

Circulating total leukocyte counts did not differ at baseline (FIG. 1G). However, with colitis ADAMTS13−/− mice had lower total circulating leukocytes compared to WT (FIG. 1G). Neutrophils increased in WT mice with colitis but were unchanged in ADAMTS13−/− mice with colitis (FIG. 1H). Lymphocytes in WT mice were unchanged, with colitis and tended to be lower in ADAMTS13−/− mice compared to WT (FIG. 1I). Monocytes rose significantly in both WT and ADAMTS13−/− mice with colitis but did not differ between the two genotypes (FIG. 1J). No significant differences in the pro-inflammatory cytokine IL-6, or the anti-inflammatory cytokine IL-10, or thrombin anti-thrombin complexes (TAT) or soluble P-selectin (sPsel) between WT and ADAMTS13−/− mice with colitis (FIG. 5A-5D).

ADAMTS13−/− Mice Develop Colonic Sub-Mucosal Thrombi with Colitis.

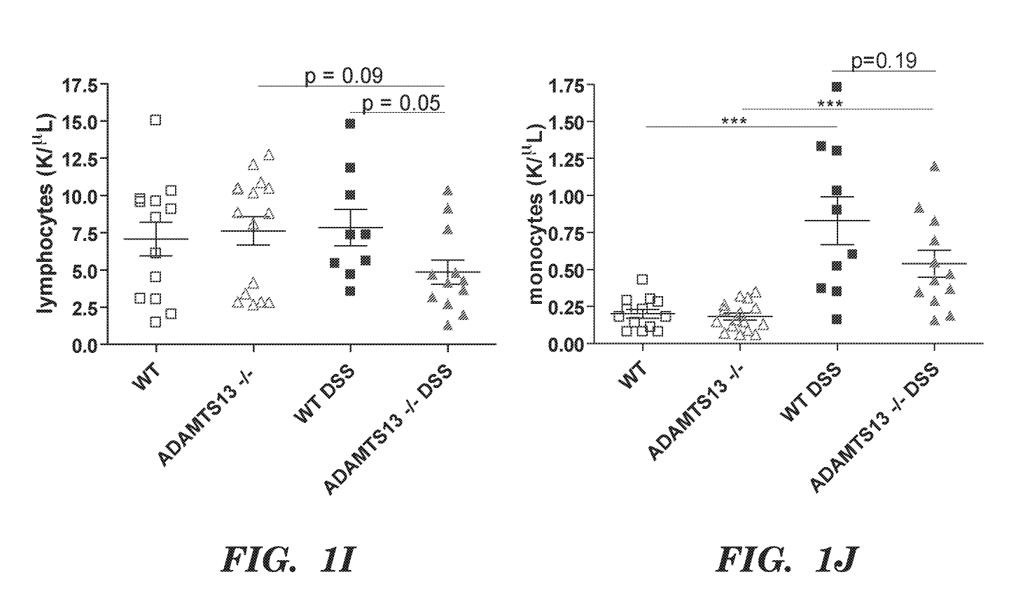

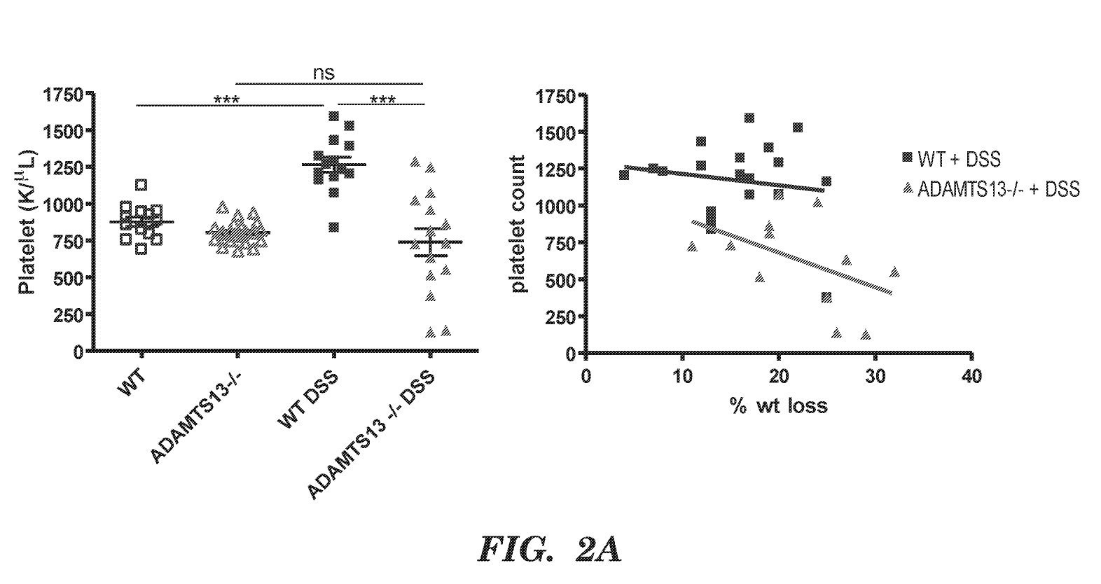

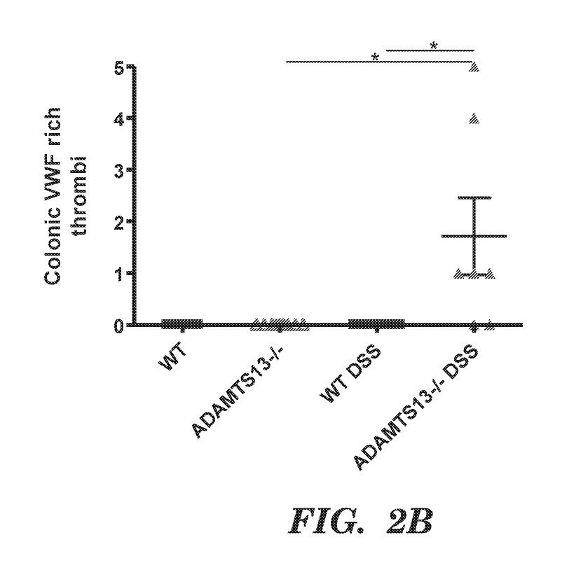

Because thrombocytosis is typically seen with colitis in both mice and humans and is even considered a marker of colonic inflammation (8, 29); platelet counts in WT and ADAMTS13−/− mice were evaluated before and after colitis initiation. Platelet counts in WT and ADAMTS13−/− mice did not differ prior to colitis initiation (FIG. 2A). WT mice demonstrated the typical pattern of significant thrombocytosis with DSS colitis (p<0.0005). Whereas ADAMTS13−/− mice had no significant difference in platelets after the development of colitis (p=0.601). In fact only those with the most severe colitis phenotype tended to be more thrombocytopenic (FIG. 2B). This finding led to testing whether in ADAMTS13−/− mice platelets were consumed in thrombi. It was hypothesized that thrombi would be at the site of inflammation in colonic vessels where ULVWF was left uncut by the lack of ADAMTS13. Entire Swiss rolled colons were immunofluorescently stained for VWF and endothelial cells with PECAM to identify vessels. In ADAMTS13−/− mice with colitis one or more sub-mucosal vessels were completely occluded by a VWF rich thrombus, compared to WT where none were found (FIG. 2B, p<0.05). Notably, in WT and ADAMTS13−/− colons from mice without colitis we found no sub-mucosal thrombi.

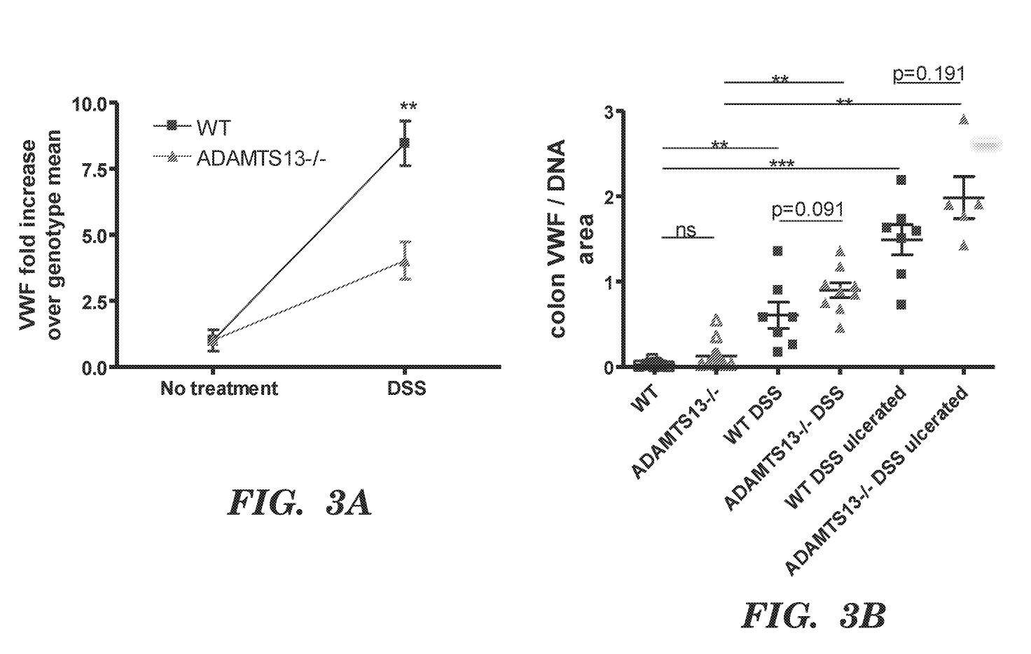

Plasma VWF was quantified at baseline in WT and ADAMTS13−/− which did not differ significantly prior to the onset of colitis. By day 10 of colitis plasma VWF increased significantly more in WT than in ADAMTS13−/− mice (FIG. 3A).

Both WT and ADAMTS13−/− Mice Show Prominent VWF Staining in Inflamed Colonic Tissue.

In immunofluorescently stained colons from WT and ADAMTS13−/− mice without colitis VWF was closely associated with PECAM-1 positive endothelium suggesting it was still contained in Weibel Palade bodies (data not shown). In contrast, both WT and ADAMTS13−/− mice with colitis had vastly increased areas of VWF stained colonic tissue extending beyond PECAM-1 positive vessels. Colonic sections were assessed at random by a reviewer blinded to mouse genotype (FIG. 3B, both p<0.005). A tendency towards more VWF remaining in the colonic tissue was seen in ADAMTS13−/− mice compared to WT (p=0.091). When ulcerated tissue was evaluated by an expert observer the strongest VWF staining was see in ADAMTS13−/− mice with colitis (data not shown). Minimal VWF staining was observed in colon tissue from an ADAMTS13−/− mouse without colitis. With colitis there is increased VWF released from endothelium in WT and more so in ADAMTS13−/− with colitis. This apparent retention of released VWF in inflamed tissue may explain the relatively lower increase of plasma VWF in colitis in the ADAMTS13−/− mice (FIG. 2A)

Increased VWF Release is Seen in Spontaneous Murine Colitis Models and in Human Colitis Specimens.

DSS colitis is produced in response to an acute colonic injury which differs immunologically from chronic colitis models and human disease where inflammation is relapses and remits. Despite this difference, it was hypothesized that increased colonic VWF release may also occur in spontaneous immune-mediated colitis models and in human colitis. Indeed, prominent VWF staining was found in colitic tissue from several spontaneous immune mediated colitis models (WASP/IL4/IL13 −/−, WASP−/−, WT CD4 (+) T cell transfer Rag2 (−/−) IL10rb (−/−), WASP−/− WT with CD4 (+) T cell transfer, Rag2 (−/−), data not shown). WT mice colon tissue sections stained for VWF and DNA show minimal VWF positive staining more so around vessels. Colon sections from T-cell transfer (DF) and spontaneous colitis models with increased VWF staining suggesting this is a phenomena seen in other models of inflammation. In human inflamed colonic tissue (data not shown), similar to DSS colitis more VWF release was seen at sites of marked inflammation in comparison to healthy sites in human colitis biopsy or resection specimens.

Treatment with rhADAMTS13 Decreases Clinical Colitis Severity and Plasma IL-6 Levels.

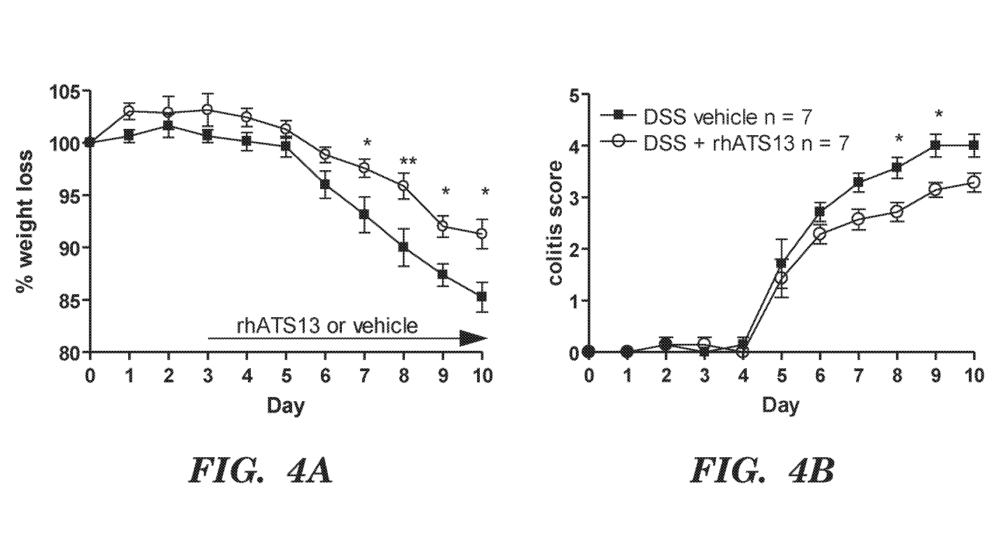

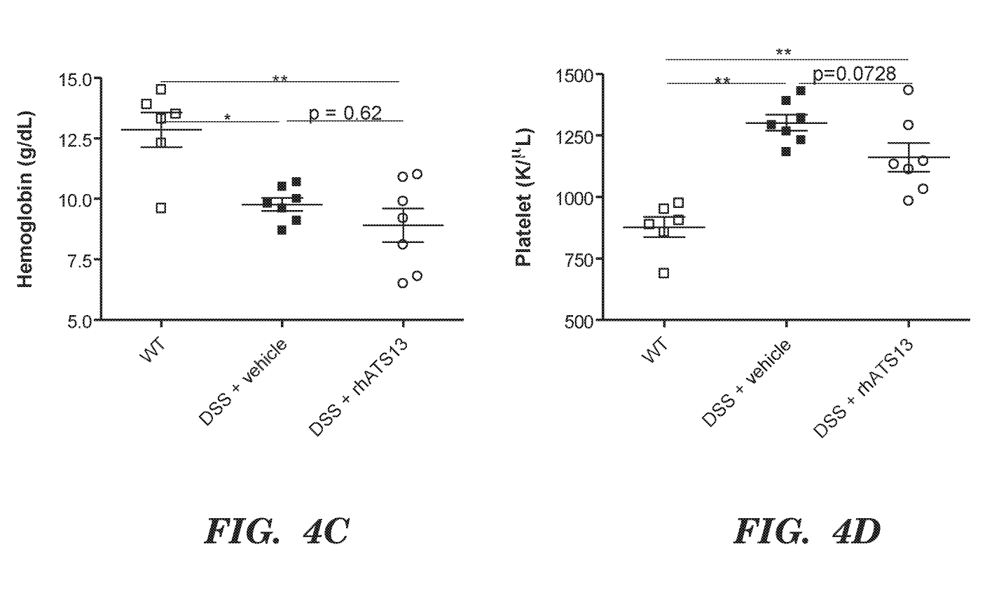

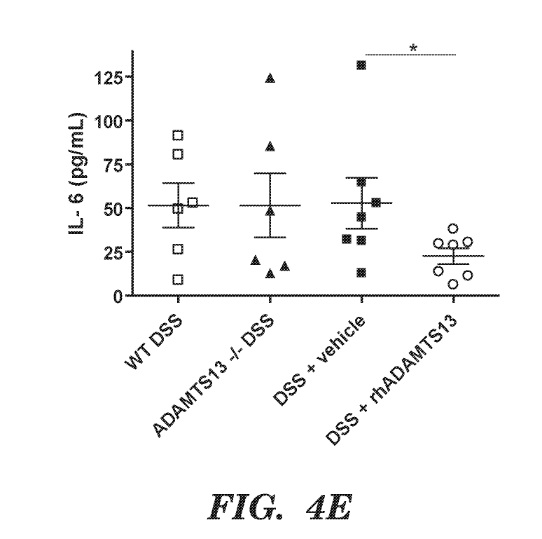

Since colitis was worse in mice in the absence of ADAMTS13, and colitic WT mice had highly increased VWF release both in plasma (FIG. 3A) and in inflamed colonic tissue (FIG. 3 B), it was hypothesized that systemic administration of rhADAMTS13 to supplement endogenous ADAMTS13 in WT mice would improve colitis. rhADAMTS13 or saline vehicle was administered intravenously daily beginning on day 3 which is two days before the onset of weight loss in the DSS model. In mice treated with rhADAMTS13, less weight loss (FIG. 4A), and less severe clinical colitis scores on days 8-10 than vehicle treated mice (FIG. 4B) were observed. While a drop in hemoglobin typical of colitis with bloody diarrhea was not observed, there was not a significant difference in hemoglobin levels between rhADAMTS13 and vehicle treated mice indicating an absence of excessive hemorrhaging as a result of treatment (FIG. 4C). The expected increase in platelet counts were observed in both rhADAMTS13 and vehicle treated groups with colitis. However, there tended to be less thrombocytosis in the rhADAMTS13 treated group (FIG. 4D). Significant differences were not found between rhADAMTS13 and vehicle treated groups with respect to colon length, scores of microscopic inflammation of H&E stained colon sections, VWF colon staining, plasma VWF or spleen weight.

IL-6 is a pathologically important cytokine in human and murine colitis. It is both pro-inflammatory and prothrombotic cytokine, which enhances platelet activation and aggregation, increases platelet leukocyte aggregates, and accelerates arteriole thrombopoeisis in DSS colitis (29). Administration of rhADAMTS13 resulted in lower IL-6 levels in plasma. This may further explain the beneficial effects of rhADAMT13 on body weight and colonic pathology of the treated mice (FIG. 4 A+4B). No differences in plasma IL10, TAT, sPsel, were detected between rhADAMTS13 and vehicle treated mice (FIGS. 5A-5D).

### DISCUSSION

The present findings demonstrate the important protective role of ADAMTS13 in colitis and provide an additional mechanism linking thrombosis and inflammation in IBD. Decreased ADAMTS13 activity in IBD can contribute to the increased rates of thrombosis in this group and also worsen colonic inflammation through decreased ULVWF cleavage and clearance from colonic vessel endothelium. Similar to the present findings in mice with colitis, submucosal thrombi are reported in rectal biopsies from patients with IBD and not in healthy controls (31). Additionally, mesenteric thrombosis is seen more often in IBD compared to controls (32). Without wishing to be bound by theory, this indicates that colonic inflammation releases ULVWF that when uncut by ADAMTS13 entangles platelets, resulting in the formation of VWF rich thrombi in submucosal vessels.

Described herein are signs of increased ULVWF release with colonic inflammation in DSS colitis, immune mediated spontaneous colitis, and in inflamed human colon tissue samples. ULVWF release was most marked at ulcerated sites. Multiple factors may contribute to endothelial activation in colitis including exposure to microbes, circulating immune complexes, localized hypoxia, activated platelets, circulating platelet leukocyte aggregates and cytokines {Bernardo, 2004 #925} which could lead to Weibel-Palade body release of ULVWF. While initially platelets prevent bleeding from vascular hemorrhaging; when ULVWF is left uncleaved by ADAMTS13 excess platelet and leukocyte recruitment worsens inflammation and can eventually lead to thrombosis {Goerge, 2008 #979}. Microvascular and submucosal thrombosis would then further worsen inflammation through tissue hypoxia, resulting ROS production, and increased leukocyte recruitment.

Plasma VWF did not differ at baseline between WT and ADAMTS13−/− mice but increased significantly more with disease progression in WT than in ADAMTS13−/− colitic mice. Without wishing to be bound by theory, it is contemplated herein that the marked increase in plasma VWF observed in WT mice compared to ADAMTS13−/− mice occurred because more VWF remained sequestered in the inflamed colons of ADAMTS13−/− mice when it is not released into circulation by ADAMTS13 cleavage. This VWF may remain attached to the inflamed vessels or to micro-thrombi formed by activated platelets. The increase in plasma VWF seen in both genotypes is likely a result of increased liberated from inflamed colonic vessels. Our findings in WT mice agree with observations in human subjects where high circulating VWF levels are found with active colitis. Circulating VWF multimers bound with activated platelets, and platelet leukocyte aggregates could also be a nidus for thrombosis at extra-colonic sites in people with active IBD. VWF thus represents an intriguing biomarker which could be measured in patients with IBD as an indicator of disease severity. It could also be used to risk stratify patients for antithrombotic treatments including rhADAMTS13 to decrease their thrombosis risk.

rhADAMTS13 treatment represents an appealing potential treatment for colitis as it decreases both inflammation and thrombosis risk. As described herein, treatment of WT mice with rhADAMTS13 decreased colitis severity by lessening weight loss, decreasing degree of anemia and thrombocytosis. Notably, despite active colonic bleeding in colitis, rhADAMTS13 treatment did not worsen anemia. Thus rhADAMTS13 administration does not lead to more severe colonic bleeding an important feature as a therapeutic in colitis. Less thrombocytosis with rhADAMTS13 treatment supports its anti-inflammatory role, as thrombocytosis is a surrogate marker of inflammation. Decreased thrombocytosis may also be a result of less severe anemia with resulting less hematopoetic growth factor production stimulating megakaryocyte proliferation. RhADAMTS13 treatment resulted in a reduction of plasma IL-6. IL-6 is a proinflammatory cytokine associated with colitis in both mice and humans (33). IL-6 also increases platelet production and these newly formed platelets are more thrombogenic (10) have increased sensitivity to platelet agonists like thrombin (34). Lower IL-6 levels are another possible reason for less thrombocytosis with rhADAMTS13 treatment. (Lower IL6 with ADAMTS13 treatment suggests an anti-inflammatory effect potentially through decreased platelet activation, decreased leukocyte adhesion to ULVWF with the resulting inflammatory cascade. sPsel and TAT levels were not significantly different between rhADAMTS13 and vehicle treated groups (data not shown). Interestingly increased IL-6 correlates with higher platelet counts suggesting a relationship between the two. Both are associated with worse disease activity in colitis (29).

The results described herein indicate that VWF and ADAMTS13 play significant roles in modulating colitis. They are of potential interest as biomarkers of inflamed endothelium, and thrombotic risk in patients with IBD. It is demonstrated herein that rhADAMTS13 had anti-inflammatory effects without worsening colonic bleeding and is a novel therapeutic target for IBD.

### REFERENCES

1. Kappelman M D, Moore K R, Allen J K, Cook S F. Recent trends in the prevalence of Crohn's disease and ulcerative colitis in a commercially insured U S population. Digestive diseases and sciences. 2013; 58(2):519-25. doi: 10.1007/s10620-012-2371-5. PubMed PMID: 22926499; PubMed Central PMCID: PMC3576554.
2. Xavier R J, Podolsky D K. Unravelling the pathogenesis of inflammatory bowel disease. Nature. 2007; 448(7152):427-34. doi: 10.1038/nature06005. PubMed PMID: 17653185.
3. Bernstein C N, Blanchard J F, Houston D S, Wajda A. The incidence of deep venous thrombosis and pulmonary embolism among patients with inflammatory bowel disease: a population-based cohort study. Thrombosis and haemostasis. 2001; 85(3):430-4. PubMed PMID: 11307809.
4. Zitomersky N L, Verhave M, Trenor C C, 3rd. Thrombosis and inflammatory bowel disease: a call for improved awareness and prevention. Inflammatory bowel diseases. 2011; 17(1):458-70. doi: 10.1002/ibd.21334. PubMed PMID: 20848518.
5. Ozturk K, Guler A K, Cakir M, Ozen A, Demirci H, Turker T, et al. Pulse Wave Velocity, Intima Media Thickness, and Flow-mediated Dilatation in Patients with Normotensive Normoglycemic Inflammatory Bowel Disease. Inflammatory bowel diseases. 2015. doi: 10.1097/MIB.0000000000000355. PubMed PMID: 25803507.
6. Ha C, Magowan S, Accortt N A, Chen J, Stone C D. Risk of arterial thrombotic events in inflammatory bowel disease. The American journal of gastroenterology. 2009; 104(6):1445-51. doi: 10.1038/ajg.2009.81. PubMed PMID: 19491858.
7. Danese S, Papa A, Saibeni S, Repici A, Malesci A, Vecchi M. Inflammation and coagulation in inflammatory bowel disease: The clot thickens. The American journal of gastroenterology. 2007; 102(1):174-86. doi: 10.1111/j.1572-0241.2006.00943.x. PubMed PMID: 17100967.
8. Harries A D, Fitzsimons E, Fifield R, Dew M J, Rhoades J. Platelet count: a simple measure of activity in Crohn's disease. British medical journal. 1983; 286(6376):1476. PubMed PMID: 6405846; PubMed Central PMCID: PMC1547707.
9. Andoh A, Yoshida T, Yagi Y, Bamba S, Hata K, Tsujikawa T, et al. Increased aggregation response of platelets in patients with inflammatory bowel disease. Journal of gastroenterology. 2006; 41(1):47-54. doi: 10.1007/s00535-005-1721-x. PubMed PMID: 16501857.
10. Esmon C T. The impact of the inflammatory response on coagulation. Thrombosis research. 2004; 114(5-6):321-7. doi: 10.1016/j.thromres.2004.06.028. PubMed PMID: 15507261.
11. Feys H B, Canciani M T, Peyvandi F, Deckmyn H, Vanhoorelbeke K, Mannucci P M. ADAMTS13 activity to antigen ratio in physiological and pathological conditions associated with an increased risk of thrombosis. British journal of haematology. 2007; 138(4):534-40. doi: 10.1111/j.1365-2141.2007.06688.x. PubMed PMID: 17608762.
12. Ley K, Laudanna C, Cybulsky M I, Nourshargh S. Getting to the site of inflammation: the leukocyte adhesion cascade updated. Nature reviews Immunology. 2007; 7(9):678-89. doi: 10.1038/nri2156. PubMed PMID: 17717539.
13. Sandborn W J, Feagan B G, Rutgeerts P, Hanauer S, Colombel J F, Sands B E, et al. Vedolizumab as induction and maintenance therapy for Crohn's disease. The New England journal of medicine. 2013; 369(8):711-21. doi: 10.1056/NEJMoa1215739. PubMed PMID: 23964933.
14. Feagan B G, Rutgeerts P, Sands B E, Hanauer S, Colombel J F, Sandborn W J, et al. Vedolizumab as induction and maintenance therapy for ulcerative colitis. The New England journal of medicine. 2013; 369(8):699-710. doi: 10.1056/NEJMoa1215734. PubMed PMID: 23964932.
15. Sporn L A, Marder V J, Wagner D D. Inducible secretion of large, biologically potent von Willebrand factor multimers. Cell. 1986; 46(2):185-90. PubMed PMID: 3087627.
16. Wagner D D. Cell biology of von Willebrand factor. Annual review of cell biology. 1990; 6:217-46. doi: 10.1146/annurev.cb.06.110190.001245. PubMed PMID: 2275814.
17. Sporn L A, Marder V J, Wagner D D. von Willebrand factor released from Weibel-Palade bodies binds more avidly to extracellular matrix than that secreted constitutively. Blood. 1987; 69(5):1531-4. PubMed PMID: 3105624.
18. Arya M, Anvari B, Romo G M, Cruz M A, Dong J F, McIntire L V, et al. Ultralarge multimers of von Willebrand factor form spontaneous high-strength bonds with the platelet glycoprotein Ib-IX complex: studies using optical tweezers. Blood. 2002; 99(11):3971-7. doi: 10.1182/blood-2001-11-0060. PubMed PMID: 12010796.
19. Dong J F, Moake J L, Nolasco L, Bernardo A, Arceneaux W, Shrimpton C N, et al. ADAMTS-13 rapidly cleaves newly secreted ultralarge von Willebrand factor multimers on the endothelial surface under flowing conditions. Blood. 2002; 100(12):4033-9. doi: 10.1182/blood-2002-05-1401. PubMed PMID: 12393397.
20. Chauhan A K, Kisucka J, Brill A, Walsh M T, Scheiflinger F, Wagner D D. ADAMTS13: a new link between thrombosis and inflammation. The Journal of experimental medicine. 2008; 205(9):2065-74. doi: 10.1084/jem.20080130. PubMed PMID: 18695007; PubMed Central PMCID: PMC2526201.
21. Stevens T R, James J P, Simmonds N J, McCarthy D A, Laurenson I F, Maddison P J, et al. Circulating von Willebrand factor in inflammatory bowel disease. Gut. 1992; 33(4):502-6. PubMed PMID: 1582595; PubMed Central PMCID: PMC1374067.
22. Zezos P, Papaioannou G, Nikolaidis N, Vasiliadis T, Giouleme O, Evgenidis N. Elevated plasma von Willebrand factor levels in patients with active ulcerative colitis reflect endothelial perturbation due to systemic inflammation. World journal of gastroenterology: WJG. 2005; 11(48):7639-45. PubMed PMID: 16437691.
23. D. Owczarek D C, K. Salapa, T. Rodacki, R. DomagalaRodacka, T. Mach, A. Undas. Decreased plasma ADAMTS13 antigen and ADAMTS13 activity as a risk factor for hypercoagulability in patients with ulcerative colitis. Journal of Crohns and Colitis. 2015; 9(suppl 1):S208-9.
24. Chauhan A K, Walsh M T, Zhu G, Ginsburg D, Wagner D D, Motto D G. The combined roles of ADAMTS13 and VWF in murine models of TTP, endotoxemia, and thrombosis. Blood. 2008; 111(7):3452-7. doi: 10.1182/blood-2007-08-108571. PubMed PMID: 18083848; PubMed Central PMCID: PMC2275014.
25. Wirtz S, Neurath M F. Mouse models of inflammatory bowel disease. Advanced drug delivery reviews. 2007; 59(11):1073-83. doi: 10.1016/j.addr.2007.07.003. PubMed PMID: 17825455.
26. Savchenko A S, Borissoff J I, Martinod K, De Meyer S F, Gallant M, Erpenbeck L, et al. VWF-mediated leukocyte recruitment with chromatin decondensation by PAD4 increases myocardial ischemia/reperfusion injury in mice. Blood. 2014; 123(1):141-8. doi: 10.1182/blood-2013-07-514992. PubMed PMID: 24200682; PubMed Central PMCID: PMC3879903.
27. Brill A, Fuchs T A, Chauhan A K, Yang J J, De Meyer S F, Kollnberger M, et al. von Willebrand factor-mediated platelet adhesion is critical for deep vein thrombosis in mouse models. Blood. 2011; 117(4):1400-7. doi: 10.1182/blood-2010-05-287623. PubMed PMID: 20959603; PubMed Central PMCID: PMC3056477.
28. Mizoguchi E. Chitinase 3-like-1 exacerbates intestinal inflammation by enhancing bacterial adhesion and invasion in colonic epithelial cells. Gastroenterology. 2006; 130(2):398-411. doi: 10.1053/j.gastro.2005.12.007. PubMed PMID: 16472595.
29. Yan S L, Russell J, Harris N R, Senchenkova E Y, Yildirim A, Granger D N. Platelet abnormalities during colonic inflammation. Inflammatory bowel diseases. 2013; 19(6):1245-53. doi: 10.1097/MIB.0b013e318281f3df. PubMed PMID: 23518812; PubMed Central PMCID: PMC3690620.
30. Senchenkova E Y, Komoto S, Russell J, Almeida-Paula L D, Yan L S, Zhang S, et al. Interleukin-6 mediates the platelet abnormalities and thrombogenesis associated with experimental colitis. The American journal of pathology. 2013; 183(1):173-81. doi: 10.1016/j.ajpath.2013.03.014. PubMed PMID: 23673000; PubMed Central PMCID: PMC3702741.
31. Dhillon A P, Anthony A, Sim R, Wakefield A J, Sankey E A, Hudson M, et al. Mucosal capillary thrombi in rectal biopsies. Histopathology. 1992; 21(2):127-33. PubMed PMID: 1505929.
32. Papay P, Miehsler W, Tilg H, Petritsch W, Reinisch W, Mayer A, et al. Clinical presentation of venous thromboembolism in inflammatory bowel disease. Journal of Crohn's & colitis. 2013; 7(9):723-9. doi: 10.1016/j.crohns.2012.10.008. PubMed PMID: 23127785.
33. Powell N, Lo J W, Biancheri P, Vossenkamper A, Pantazi E, Walker A W, et al. Interleukin-6 Increases Production of Cytokines by Colonic Innate Lymphoid Cells in Mice and Patients with Chronic Intestinal Inflammation. Gastroenterology. 2015. doi: 10.1053/j.gastro.2015.04.017. PubMed PMID: 25917784.
34. Burstein S A. Cytokines, platelet production and hemostasis. Platelets. 1997; 8(2-3):93-104. doi: 10.1080/09537109709169324. PubMed PMID: 20297930.

1. A method of treating an inflammatory bowel disease (IBD), the method comprising administering ADAMTS13 to a subject in need thereof.

2. The method of claim 1, wherein the method comprises treating a flare-up of an inflammatory bowel disease (IBD).

3. The method of claim 1, wherein the method comprises treating a symptom of an inflammatory bowel disease (IBD).

4. The method of claim 3, wherein the symptom of inflammatory bowel disease (IBD) is inflammation of the colon or small intestine.

5. A method of preventing a thrombosis, ischemia, and/or pulmonary embolism associated with IBD, the method comprising administering ADAMTS13 to a subject in need thereof.

6. The method of claim 1, wherein the ADAMTS13 is administered intravenously or subcutaneously.

7. (canceled)

8. The method of claim 1, wherein the ADAMTS13 is a human ADAMTS13 polypeptide.

9. The method of claim 1, wherein the ADAMTS13 is recombinant ADAMTS13.

10. The method of claim 1, wherein the ADAMTS13 is plasma-derived ADAMTS13.

11. The method of claim 1, wherein the ADAMTS13 is administered at a dosage of from about 20 U/kg to about 1000 U/kg.

12. (canceled)

13. The method of claim 1, wherein the ADAMTS13 is administered at a dosage of from about 40 U/kg to about 250 U/kg.

14. The method of claim 1, wherein the ADAMTS13 is administered at least every 2 days.

15. The method of claim 1, wherein the ADAMTS13 is administered daily.

16. The method of claim 1, further comprising:
(a) determining a level of ADAMTS13 expression or activity in the subject; and
(b) administering ADAMTS13 to the subject if the level of ADAMTS13 expression or activity is reduced relative to the average level of ADAMTS13 expression in subjects without inflammatory bowel disease.

17. (canceled)

18. The method of claim 1, wherein the subject is administered ADAMTS13 in response to a flare-up of the IBD.

19. The method of claim 1, wherein the inflammatory bowel disease (IBD) is selected from the group consisting of: Crohn's disease, ulcerative colitis, an idiopathic colitis, an iatrogenic colitis, ischemic colitis, infectious colitides, and eosinophilic colitis.

20. The method of claim 19, wherein the idiopathic colitis is microscopic colitis, lymphocytic colitis, or collagenous colitis.

21. The method of claim 19, wherein the iatrogenic colitis is diversion colitis, neutropenic enterocolitis, disinfectant colitis, corrosive colitis, nonsteroidal anti-inflammatory drug and salicylate-induced colitis, toxic epidermal necrolysis, or another chemical-induced colitis

22. The method of claim 19, wherein the infectious colitis is Clostridium difficile colitis.

23. A composition for use in the treatment of an inflammatory bowel disease (IBD), the composition comprising ADAMTS13.

24.-62. (canceled)


SEQUENCE LISTING (2 sequences)

SEQ ID NO: 1 - DNA, 4966 bp, Homo sapiens
aaccacgatgtctttggcacagcctctcatctgtcagatgggagcggggaccccggagagggagtcagccgaggtcctggcattccttgtgaacccccgtctgtgggtttctggtccagtgtcccttctccagattagatggcttaggcctcctctaagggggtgggcgtgcacatccggagagctgtctggtgtgcaggactgggctgcaggttaccctgaactgcaaccatcttagagcaaggcccagcttgcagcaggaggagctgcaggccgcccaccctagccacggcccctgccctggcaggaagcttccaagagtaaacactgcctaatcgtcccgcccagtagtgagcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggtggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtacgcccagcctggtgccccacgaagaagccgctgctccaggacggaccacagccacccctgctggtgcctccctggagtggtcccaggcccggggcctgctcttctccccggctccccagcctcggcggctcctgcccgggccccaggaaaactcagtgcagtccagtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctgagggtcattgaacatttgttccgtgtctggccagccctggagggttgacccctggtctcagtgctttccaattcgaactttttccaatcttaggtatctactttagagtcttctccaatgtccaaaaggctagggggttggaggtggggactctggaaaagcagcccccatttcctcgggtaccaataaataaaacatgcaggctgaccggcgtttttttcttaaaaaaaaaaaaaaaa

SEQ ID NO: 2 - protein, 1427 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT